(19) United States
(12) Patent Application Publication
(10) Pub. No.: US 2024/0011088 A1
(43) Pub. Date: Jan. 11, 2024

(54) METHODS AND DEVICES FOR SPATIALLY RESOLVED ANALYSIS OF PROTEOMIC AND GENETIC INFORMATION

(71) Applicant: IsoPlexis Corporation, Branford, CT (US)

(72) Inventors: Rui ZHENG, Madison, CT (US); Sean MACKAY, New Haven, CT (US)

(21) Appl. No.: 18/235,535

(22) Filed: Aug. 18, 2023

Related U.S. Application Data
(63) Continuation of application No. PCT/US2022/016978, filed on Feb. 18, 2022.
(60) Provisional application No. 63/151,182, filed on Feb. 19, 2021.

(51) Int. Cl. C12Q 1/6869; C12Q 1/686

(52) U.S. Cl. CPC C12Q 1/6869; C12Q 1/686

(57) ABSTRACT

Disclosed are devices and methods capable of determining spatially resolved information from a biological sample including genomic, transcriptomic, and proteomic information.

### RELATED APPLICATIONS

This application is a continuation of International Application No. PCT/US2022/016978, filed on Feb. 18, 2022, which claims the benefit of U.S. Application No. 63/151,182, filed on Feb. 19, 2021, the entire contents of each which are herein incorporated by reference in their entireties.

### FIELD OF THE DISCLOSURE

The disclosure is directed to molecular biology, and more specifically to devices and methods of determining spatially resolved proteomic, transcriptomic, and genomic information in biological samples.

### BACKGROUND

There are long felt but unmet needs in the art for devices and methods capable of determining spatially resolved information in biological samples, including the ability to simultaneously analyze proteomic, transcriptomic, and genomic information from a sample and to understand the spatial distribution of these analytes across the sample. This disclosure provides devices and methods capable of analyzing proteomic, transcriptomic, and genomic information in biological samples, such as organ and tumor tissue, in a high-throughput format wherein hundreds of intracellular components, including DNA, RNA, and protein levels, are analyzed in parallel. The disclosure provides devices and methods to solve these long felt but unmet needs.

### SUMMARY

According to some embodiments, a multiplex assay chip device is provided which is configured for multiplexed analysis of biological material, the device comprising a plurality of capture beads (CB), each bead including a CB capture moiety and having a diameter, a substrate having a plurality of chambers, each of the plurality of chambers including an open end arranged on a first side of the substrate, at least one first region having a first length, a first width, and a first depth, wherein each of the first length, the first width, and the first depth is greater than the bead diameter, at least one second region having a second length, a second width, and a second depth, wherein at least one of the second length, the second width, and the second depth is less than the bead diameter, at least one CB arranged within the at least one first region of each of the plurality of chambers of the substrate, and a surface removably couplable to the first side of the substrate, wherein each of the plurality of chambers is covered when the surface is removably coupled to the first side of the substrate.

The above-noted embodiments may further include one and/or another (and in some embodiments, a plurality of, in some embodiments, a majority of, in some embodiments, substantially all of, and in some embodiments, all of) of the following features, functionality, steps, material, compositions, components, and/or clarifications, yielding yet further embodiments of the disclosure:
at least one substrate capture moiety attached to a surface of the at least one second region;
at least one first region is two first regions and each one of the two first regions have different dimensions;
the at least one second region has a width of between 1 μm and 100 μm;
the at least one second region has a length of between 1 μm and 2000 μm;
the at least one second region has a depth of between 1 μm and 100 μm;
the distance between adjacent ones of the plurality of chambers is between 0.01 μm and 10 μm;
the first width of the at least one first region is between 5 μm and 50 μm larger than the width of the second width of the at least one second region;
the at least one first region is cylindrical and each of the first length and the first width are between 10 and 100 μm;
a second one of the two first regions has a dimension between 5 μm and 50 μm larger than a corresponding dimension of the first one of the first wo regions;
the second one of the two first regions has a dimension of between 15 μm and 150 μm;
a diameter of a CB capture moiety within the first one of the two first regions is between 0 μm and 50 μm smaller than a dimension of the first one of the two first regions;
a diameter of a CB capture moiety within the second one of the two first regions is between 0 μm and 50 μm smaller than a dimension of the second one of the two first regions;
the diameter of the CB capture moiety within the first one of the two first regions is larger than the diameter of the CB capture moiety within the second one of the two first regions;
a cross-section of the at least one first region is one or more of circular, ovoid, rectangular, square, triangular, pentagonal, hexagonal, and octagonal;
the CB capture moiety is an oligonucleotide capture bead comprising a nucleic acid capture sequence tethered to the CB;
the nucleic acid capture sequence of the CB capture moiety comprises an individually unique chamber barcode sequence, a PCR handle, a unique molecular identifier (UMI), a barcode handle sequence, and a capture sequence;
each nucleic acid capture sequence of the CB capture moiety comprises a unique UMI;
the barcode sequence of the CB capture moiety is unique to each chamber of the plurality of chambers;
the at least one substrate capture moiety is an antibody;
the CB capture moiety comprises an antibody tethered to the CB;
the surface comprises glass;
the plurality of chambers comprises between 10,000 chambers and 100,000 chambers;
the substrate comprises a polymer; and/or
the polymer comprises polydimethylsiloxane.

According to some embodiments of the present disclosure, a method is provided for determining spatially resolved information from a biological sample, comprising (a) obtaining a device according to embodiments disclosed herein, (b) removing the surface from the substrate, (c) mounting a histological sample to the surface, (d) treating the histological sample with a cell lysis or permeabilization reagent, under conditions sufficient for target biological molecules to be released from the histological sample, (e) coupling the surface to the substrate to cover the plurality of chambers, wherein the histological sample is disposed between the substrate and the surface, and wherein the surface seals the plurality of chambers forming a plurality of enclosures that are fluidicly isolated from each other, (f) incubating the histological sample under conditions sufficient for the target biological molecules to form complexes with the CB capture moiety and the substrate capture moiety, and (g) detecting the complexes.

According to some embodiments of the present disclosure, a method is provided for determining spatially resolved information from a biological sample, comprising I) providing a device comprising a substrate having a plurality of chambers each comprising at least one first capture bead (CB), II) obtaining a sectioned histological sample from a subject mounted to a surface, III) treating the sectioned histological sample with a cell lysis or permeabilization reagent, under conditions sufficient for target biological molecules to be released from the sample, IV) coupling the surface to the substrate such that the surface seals the plurality of chambers forming a plurality of enclosures; wherein each enclosure comprises a first CB in fluid communication with a portion of the sectioned histological sample and target biological molecules, V) incubating the sample under conditions sufficient for the target biological molecules to contact the CB to form CB-target biological molecule complexes, and VI) detecting the complexed target biological molecule.

According to some embodiments of the present disclosure, a method is provided for determining spatially resolved information from a biological sample, comprising I) providing a device comprising a substrate having a plurality of chambers each comprising at least one first capture bead (CB), II) obtaining a sectioned histological sample from a subject mounted to a surface, III) treating the sectioned histological sample with a cell lysis or permeabilization reagent, under conditions sufficient for target nucleic acid sequences to be released from the sample, IV) coupling the surface to the substrate such that the surface seals the plurality of chambers forming a plurality of enclosures; wherein each enclosure comprises a first CB in fluid communication with a portion of the sectioned histological sample and target nucleic acid sequences, V) incubating the sample under conditions sufficient for the target nucleic acid sequences to contact the CB to form CB-target nucleic acid sequence complexes, and VI) sequencing the complexed target nucleic acid sequences.

The above-noted embodiments may further include one and/or another (and in some embodiments, a plurality of, in some embodiments, a majority of, in some embodiments, substantially all of, and in some embodiments, all of) of the following features, functionality, steps, material, compositions, components, and/or clarifications, yielding yet further embodiments of the disclosure:
the biological sample is a frozen or formaldehyde-fixed sectioned histological sample;
the sectioned histological sample is stained with H&E stain or an immunofluorescence stain;
the immunofluorescence stain is specific for a biomarker or cellular organelle;
the method further comprises imaging the stained sectioned histological sample;
the imaging occurs prior to treating the sectioned histological sample with the cell permeabilization reagent;
the sectioned histological sample is healthy tissue, cancerous tissue, a tumor, an organ, blood, or an embryo;
the cell permeabilization reagent is a cell lysis reagent;
target biological molecules comprise at least one target protein or at least one target nucleic acid sequence;
the target biological molecules comprise at least one target protein and at least one target nucleic acid sequence;
the at least one target nucleic acid sequence is an RNA sequence;
the RNA sequence is an mRNA sequence;
the first CB is configured to capture nucleic acid sequences, peptides, proteins, metabolites, or organic molecules;
the first CB comprises a capture moiety configured to capture nucleic acid sequences;
the first CB comprises a capture moiety configured to capture proteins;
the method further comprises at least one second capture bead;
the second CB is configured to capture nucleic acid sequences, peptides, proteins, metabolites, or organic molecules;
the second CB comprises a capture moiety configured to capture nucleic acid sequences;
the second CB comprises a capture moiety configured to capture proteins;
each chamber of the plurality of chambers comprises a first CB and a second CB;
the first CB and the second CB are configured to capture different biological molecules;
the target nucleic acid sequences comprise from about one to about 1,000,000 target nucleic acid sequences;
the target protein comprises from one to about 1,000,000 target proteins;
the CB comprises a plurality of nucleic acid sequences, each comprising an individually unique barcode sequence comprising a predetermined number of base pairs, a PCR handle, a unique molecular identifier (UMI), a barcode handle sequence, and a capture sequence;
the CB comprises from one to 10,000,000 nucleic acid sequences;
the barcode sequence of the CB is unique to each CB of the plurality of CBs;
the barcode sequence of the CB is unique to each chamber of the plurality of chambers;
each nucleic acid sequence of the CB comprises a unique UMI;
the individually unique barcode sequence of the CB is sequenced;
sequencing the individually unique barcode sequence comprises synthesizing a cDNA barcode sequence;
synthesizing the cDNA barcode sequence comprises contacting the sequence encoding the barcode handle with a primer comprising a sequence complementary to a portion of the sequence encoding the barcode handle and a polymerase, under conditions sufficient for hybridization and cDNA synthesis;
the contacting produces a cDNA comprising a cDNA barcode sequence;
an individually unique chamber barcode sequence of the CB is sequenced;
sequencing the individually unique chamber barcode sequence comprises synthesizing a cDNA barcode sequence;
synthesizing the cDNA barcode sequence comprises contacting the sequence encoding a barcode handle with a primer comprising a sequence complementary to a portion of the sequence encoding the barcode handle and a polymerase, under conditions sufficient for hybridization and cDNA synthesis;
the contacting produces a cDNA comprising a cDNA barcode sequence;
the sequence encoding the barcode comprises 12 nucleotides;
the conditions sufficient for hybridization and cDNA synthesis comprise a plurality of deoxynucleotides (dNTPs);
at least one dNTP of the plurality of deoxynucleotides (dNTPs) comprises a modification;
each dNTP of the plurality of deoxynucleotides (dNTPs) comprises a modification;
the modification comprises a label;
the label comprises a fluorophore or a chromophore;
the label is a fluorescent label;
each adenine comprises a first label;
each cytosine comprises a second label;
each guanine comprises a third label;
each thymine comprises a fourth label;
the first label, the second label, the third label, and the fourth label are distinct labels;
the first label, the second label, the third label, and the fourth label are spectrally distinguishable fluorescent labels;
the nucleic acid encoding the barcode further comprises a sequence encoding a template switch oligonucleotide (TSO) hybridization site;
the sequence encoding a TSO hybridization site comprises a poly-riboguanine (poly-rG) sequence;
the method further comprises contacting the nucleic acid sequence encoding the barcode of the CB and a TSO under conditions sufficient for hybridization of the TSO to a portion of the nucleic acid encoding the barcode to produce a nucleic acid/TSO duplex;
the TSO comprises a sequence complementary to the sequence encoding the UMI, a sequence complementary to the sequence encoding the TSO handle, a sequence complementary to the sequence encoding the sequence encoding a TSO hybridization site, and a sequence complementary to the target nucleic acid sequences;
sequencing comprises synthesizing a cDNA sequence comprising one of the complexed target nucleic acid sequences for each of the complexed target nucleic acid sequences;
the cDNA sequence comprises the target nucleic acid sequence, UMI, and individually unique chamber barcode sequence;
sequencing comprises removing the cDNA sequences from the chamber;
sequencing comprises amplifying the cDNA sequences by PCR;
the sequencing method is next generation sequencing (NGS);
the method further comprises analyzing the cDNA sequences and/or analyzing the cDNA sequences;
the cDNA sequences are clustered by barcode sequence;
the cDNA sequences are quantified by bioanalyzer;
the target protein contacts the bead to form a CB-target protein complex;
the detecting the target protein comprises contacting the CB-target protein complex with a labeled secondary antibody and imaging the labeled secondary antibody;
the spatial resolution is 0.1 μm to 100 μm; and/or
the spatial resolution is single cell.

### BRIEF DESCRIPTION OF THE DRAWINGS

FIG. 1 is an illustration of a spatial distribution of information within a biological tissue.

FIG. 2A is a schematic depiction of a chamber in a multiplex assay chip device, according to an embodiment. The multiplex assay chip device can be used for multiplexed analysis of biological material. Chamber C has at least one first region and at least one second region. The at least one first region may include pockets P(1) and P(2) separated by a channel c of the at least one second region. Each of the pockets P(1) and P(2) is able to have a capture bead disposed therein.

FIG. 2B is a schematic depiction of a chamber in a multiplex assay chip device, according to an embodiment. The multiplex assay chip device can be used for multiplexed analysis of biological material. Chamber C has at least one first region and at least one second region. The at least one first region may include pockets P(1), P(2), and P(3) separated by sections of a channel c, each of the sections of the channel c being one of at least one second region.

FIG. 3A is a schematic depiction of a chamber in a multiplex assay chip device, according to an embodiment. The multiplex assay chip device can be used for multiplexed analysis of biological material. Chamber C has at least one first region and at least one second region. The at least one first region may include a pocket P(1) and the at least one second region may be a channel c.

FIG. 3B is a schematic depiction of various chamber configurations in a multiplex assay chip device(s), according to an embodiment. The multiplex assay chip device(s) can be used for multiplex analysis of biological material. The chamber C has at least one first region. The at least one first region may be a pocket P(1), P(2), P(3), and/or P(4).

FIG. 4A is a schematic depiction of a chamber in a multiplex assay chip device, according to an embodiment. The multiplex assay chip device can be used for multiplexed analysis of biological material. Chamber C has at least one first region and at least one second region. The at least one first region may be pockets P(1) and P(2) separated by a channel c (i.e., the at least one second region), the pockets P(1) and P(2) of the at least one first region having capture beads CB(1) and CB(2) disposed therein.

FIG. 4B is a schematic depiction of a chamber in a multiplex assay chip device, according to an embodiment. The multiplex assay chip device can be used for multiplexed analysis of biological material. Chamber C has at least one first region and at least one second region. The at least one first region may be a pocket P(1) and the at least one second region may be a channel c connected to the pocket P(1). Pocket P(1) may have a capture bead CB disposed therein.

FIG. 4C is a schematic depiction of various chamber configurations in a multiplex assay chip device(s), according to an embodiment. The multiplex assay chip device(s) can be used for multiplexed analysis of biological material. The chamber C may be a pocket P(1), P(2), P(3), and/or P(4). Pockets P(1), P(2), P(3), and/or P(4) may have a capture bead CB disposed therein. In another regard, FIG. 4C is a schematic depiction of a non-limiting subset of shapes of pockets that may be found in any multiplex assay chip device, according to embodiments of the disclosure.

FIG. 5 is a schematic detailing the creation of a nucleic acid capture bead of the disclosure.

FIG. 6 is an image of a capture bead comprising from 5′ to 3′ a 5T spacer sequence 102, a PCR handle sequence 103, a unique molecular identifier (UMI) sequence 104, chamber barcode sequence 105 (individually unique chamber barcode), and a sequencing (seq) handle 106, the capture bead being configured to capture mRNA sequences by hybridizing a polyT capture sequence 110 to the 3′ polyA sequence of an mRNA.

FIG. 7 is a schematic detailing 5′ mRNA capture using capture beads of the disclosure configured to comprise rGrGrG (three riboguanosines) for capture of 5′ mRNA sequences that have been reverse transcribed to include a terminal polyC sequence.

FIG. 8 is an image of a capture bead configured to capture nucleic sequences by hybridizing a sequence specific capture sequence 112 to a portion of a target nucleic acid 113, such as an RNA or DNA sequence. In some embodiments, this is a gene-specific capture sequence.

FIG. 9A is a schematic detailing a device and method for determining spatially resolved information from a sectioned histological sample.

FIG. 9B is a schematic of a multiplex assay chip device having chambers of FIG. 3A disposed therein, according to an embodiment.

FIG. 10 is a schematic detailing a device and method for determining spatially resolved information from a sectioned histological sample.

FIG. 11 is a schematic depicting an exemplary workflow for determining spatially resolved information from a histological sample.

FIG. 12 is an exemplary workflow for determining spatially resolved information from a histological sample.

### DETAILED DESCRIPTION

The disclosure provides methods of performing spatially resolved analysis of biological material in biological samples.

The distribution of biological material, such as biomolecules including proteins, metabolites, and nucleic acids, across an organism is highly heterogeneous and variable over time. This heterogeneity is not limited to changes in distribution between organs or cell type. In fact, changes in the distribution of biomolecules can be observed within small subsections of organs or across sections of a tumor. This distribution can also be observed from one single cell to another single cell, even in single cells directly adjoining one another in a tissue, organ, or tumor. Methods capable of detecting this heterogeneity of expression in a spatially resolved fashion can provide critical insight into the biology of an organism including the development or progression of diseases, such as neurodegenerative diseases or cancer. Spatially resolved methods of analyzing biological molecules in a biological sample enable increased understanding of development and progression of disease, thereby offering the potential development of new therapeutic strategies for combatting disease.

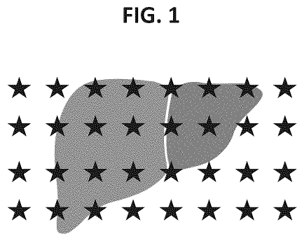

This disclosure provides devices and methods capable of determining spatially resolved information in biological samples including the ability to simultaneously analyze proteomic, transcriptomic, and genomic information from a sample and to understand the spatial distribution of these analytes across the sample, such as across a sectioned histological sample of a tumor or organ. Such a section histological sample of a tumor or organ may be a hepatic sample, as shown in FIG. 1, where stars represent a spatial distribution of proteomic, transcriptomic, and genomic information that can be determined via devices and methods of the disclosure.

### Devices and Arrays

Devices and arrays are provided and configured for use in methods of analyzing multiple cellular activities and pathways in single cells in a high-throughput format wherein hundreds of intracellular components, including DNA, RNA, and protein levels of each single cell of a plurality of thousands of cells are analyzed in parallel.

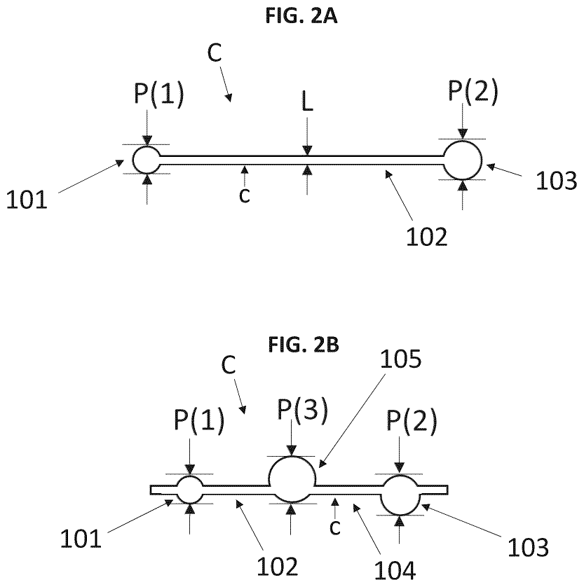

FIG. 2A through FIG. 4C provide depictions of chambers in a multiplex assay chip device, according to embodiments. The multiplex assay chip device can be used for multiplexed analysis of biological material. The multiplex assay chip device can include plurality of chambers such as chamber C shown in FIG. 2A having at least one first region 101, 103 and at least one second region 102. In an embodiment, the at least one first region 101, 103 may be two first regions having a first pocket P(1) and a second pocket P(2). The first pocket P(1) and the second pocket P(2) may be separated by the at least one second region 102 defined by channel c.

FIG. 2B is a schematic depiction of a chamber in a multiplex assay chip device, according to an embodiment. The multiplex assay chip device can be used for multiplexed analysis of biological material. The multiplex assay chip device includes a plurality of chambers such as chamber C shown in FIG. 2B having at least one first region 101, 103, 105 and at least one second region 102, 104. In an embodiment, the at least one first region 101, 103, 105 may be three regions having a first pocket P(1), a second pocket P(2), and a third pocket P(3).

As shown in FIG. 2B, the first pocket P(1) is center aligned with a channel c of the chamber C, while pockets P(2) and P(3) are not center aligned with the channel c of the chamber C. The channel c of the chamber C does not intersect with a central axis of the pockets P(3) and P(2). In some embodiments, a non-center aligned pocket can improve signal to noise ratio due to lower auto fluorescent background further from the central axis of the chamber. In some embodiments, a non-center aligned pocket can allow for increased contact between target biological molecules and capture arrays.

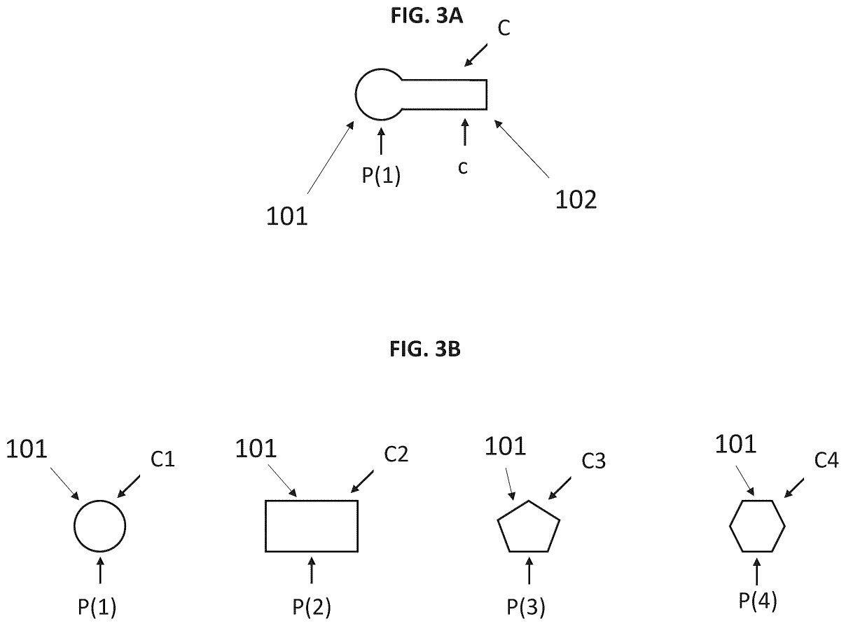

FIG. 3A is a schematic depiction of a chamber in a multiplex assay chip device, according to an embodiment. The multiplex assay chip device can be used for multiplexed analysis of biological material. The multiplex assay chip device includes a plurality of chambers such as chamber C having at least one first region 101 and at least one second region 102. In an embodiment, the at least one first region 101 may be a single region having a pocket P(1) and the at least one second region 102 may be a single region.

FIG. 3B is a schematic description of four chamber configurations, according to embodiments. Each multiplex assay chip device can be used for multiplexed analysis of biological material. Each multiplex assay chip device includes a plurality of chambers, such as chambers C1, C2, C3, or C4, or combinations thereof, having at least one first region 101. In an embodiment, the at least one first region 101 may be a single region having a pocket P(1), P(2), P(3), or P(4).

As shown in FIG. 3B, and as will be described later, the at least one first region 101 may have any cross-sectional shape, including one of a circular cross section, a rectangular cross section, a square cross section, a pentagonal cross section, an elliptical cross section, a hexagonal cross section, or any other suitable cross-sectional shape.

As shown in FIG. 3A, and as will be described later, the at least one second region 102 may have any cross-sectional shape, including one of a circular cross section, a rectangular cross section, a square cross section, a pentagonal cross section, an elliptical cross section, a hexagonal cross section, or any other suitable cross-sectional shape.

In some embodiments, the chamber C can include an open end on a first side of the multiplex assay chip device. In some embodiments, the open end of the chamber C can be physically coupled to a central atrium. In some embodiments, a surface can cover the open end of the chamber C, fluidically isolating the chamber C from the rest of the multiplex assay chip device (which may include a plurality of other chambers). The surface can cover the open end of the chamber C when, for instance, a sample mounted to the surface is exposed to the chamber C. In some embodiments, pockets of at least one first region of the chamber C can be connected and/or arranged along the channel c. In some embodiments, the chamber C and pockets of the at least one first region of the multiplex assay chip device can be included in a substrate. In some embodiments, the substrate can be at least partially composed of a polymer. In some embodiments, the polymer can include polydimethylsiloxane (PDMS).

In view of FIG. 2A through FIG. 3B, the chamber C will now be described in greater detail.

In some embodiments, the chamber C can be composed of glass, polymers, metal, silica, or any other suitable material. In some embodiments, each of the chamber C can include an open end on a first side of the multiplex assay chip device. In some embodiments, each of the open ends of the chamber C can be fluidically coupled to a central atrium. In some embodiments of systems with multiple chambers, a surface can cover the open ends of either of the chambers, fluidically isolating the chambers from one another.

In some embodiments, the chamber C can have a length of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, or at least about 1,000 μm, or at least about 2000 μm. In some embodiments, the chamber C can have a length of no more than about 2,000 μm, no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced lengths of the chamber C are also possible (e.g., at least about 1 μm and no more than about 2,000 μm or at least about 5 μm and no more than about 200 μm) inclusive of all values and ranges therebetween. In some embodiments, the chamber C can have a length of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1,000 μm, or about 2,000 μm.

In some embodiments of systems with multiple chambers, the chambers can have uniform lengths. In other words, a first chamber can have a length the same or substantially similar to a second chamber. In some embodiments, the chambers can have different lengths. In other words, a first chamber can have a length different from the length of a second chamber.

In some embodiments, the at least one first region of the chamber C can have a length of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, or at least about 1,000 μm, or at least about 2000 μm. In some embodiments, the at least one first region of the chamber C can have a length of no more than about 2,000 μm, no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced lengths of the at least one first region of the chamber C are also possible (e.g., at least about 1 μm and no more than about 2,000 μm or at least about 5 μm and no more than about 200 μm) inclusive of all values and ranges therebetween. In some embodiments, the at least one first region of the chamber C can have a length of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1,000 μm, or about 2,000 μm.

In some embodiments of systems with multiple chambers, the at least one first region of respective chambers can have uniform lengths. In some embodiments, the at least one first region of respective chambers can have different lengths.

In some embodiments, the at least one second region of the chamber C can have a length of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, or at least about 1,000 μm, or at least about 2000 μm. In some embodiments, the at least one second region of the chamber C can have a length of no more than about 2,000 μm, no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced lengths of the at least one second region of the chamber C are also possible (e.g., at least about 1 μm and no more than about 2,000 μm or at least about 5 μm and no more than about 200 μm) inclusive of all values and ranges therebetween. In some embodiments, the at least one second region of the chamber C can have a length of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1,000 μm, or about 2,000 μm.

In some embodiments of systems with multiple chambers, the at least one second region of respective chambers can have uniform lengths. In some embodiments, the at least one second region of respective chambers can have different lengths.

In some embodiments, the chamber C can have a width of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, or at least about 400 μm. In some embodiments, the chamber C can have a width of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced widths of the chamber C are also possible (e.g., at least about 1 μm and no more than about 500 μm or at least about 5 μm and no more than about 200 μm) inclusive of all values and ranges therebetween. In some embodiments, the chamber C can have a width of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm.

In some embodiments of systems with multiple chambers, the chambers can have uniform widths. In other words, a first chamber can have a width the same or substantially similar to a second chamber. In some embodiments, the chambers can have different widths. In other words, a first chamber can have a width different from the width of a second chamber.

In some embodiments, the at least one first region of the chamber C can have a width of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, or at least about 400 μm. In some embodiments, the at least one first region of the chamber C can have a width of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced widths of the at least one first region of the chamber C are also possible (e.g., at least about 1 μm and no more than about 500 μm or at least about 5 μm and no more than about 200 μm) inclusive of all values and ranges therebetween. In some embodiments, the at least one first region of the chamber C can have a width of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm.

In some embodiments of systems with multiple chambers, the at least one first region of respective chambers can have uniform widths. In some embodiments, the at least one first region of respective chambers can have different widths.

In some embodiments, the at least one second region of the chamber C can have a width (denoted as L in FIG. 2A) of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, or at least about 400 μm. In some embodiments, the at least one second region of the chamber C can have a width of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced widths of the at least one second region of the chamber C are also possible (e.g., at least about 1 μm and no more than about 500 μm or at least about 5 μm and no more than about 200 μm) inclusive of all values and ranges therebetween. In some embodiments, the at least one second region of the chamber C can have a width of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm.

In some embodiments of systems with multiple chambers, the at least one second region of respective chambers can have uniform widths. In some embodiments, the at least one second region of respective chambers can have different widths.

In some embodiments, the chamber C can have a depth of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, or at least about 400 μm. In some embodiments, the chamber C can have a depth of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced depths of the chamber C are also possible (e.g., at least about 1 μm and no more than about 500 μm or at least about 5 μm and no more than about 200 μm) inclusive of all values and ranges therebetween. In some embodiments, the chamber C can have a depth of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm.

In some embodiments of systems with multiple chambers, the chambers can have uniform depths. In other words, a first chamber can have a depth the same or substantially similar to a second chamber. In some embodiments, the chambers can have different lengths. In other words, a first chamber can have a depth different from the depth of a second chamber.

In some embodiments, the at least one first region of the chamber C can have a depth of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, or at least about 400 μm. In some embodiments, the at least one first region of the chamber C can have a depth of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced depths of the at least one first region of the chamber C are also possible (e.g., at least about 1 μm and no more than about 500 μm or at least about 5 μm and no more than about 200 μm) inclusive of all values and ranges therebetween. In some embodiments, the at least one first region of the chamber C can have a depth of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm.

In some embodiments of systems with multiple chambers, the at least one first region of respective chambers can have uniform depths. In some embodiments, the at least one first region of respective chambers can have different lengths.

In some embodiments, the at least one second region of the chamber C can have a depth of at least about 1 μm, at least about 2 μm, at least about 3 μm, at least about 4 μm, at least about 5 10 μm, at least about 6 μm, at least about 7 μm, at least about 8 μm, at least about 9 μm, at least about μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, or at least about 400 μm. In some embodiments, the at least one second region of the chamber C can have a depth of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced depths of the at least one second region of the chamber C are also possible (e.g., at least about 1 μm and no more than about 500 μm or at least about 5 μm and no more than about 200 μm) inclusive of all values and ranges therebetween. In some embodiments, the at least one second region of the chamber C can have a depth of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm.

In some embodiments of systems with multiple chambers, the at least one second region of respective chambers can have uniform depths. In some embodiments, the at least one second region of respective chambers can have different lengths.

In some embodiments, the chambers can positioned such that the distance between chambers is at least about 0.00001 μm, at least about 0.0001 μm, at least about 0.001 μm, at least about 0.01 μm, at least about 0.1 μm, at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1,000 μm, or at least about 2,000 μm. In some embodiments, the distance between chambers can be no more than about 2,000 μm, no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, no more than about 2 μm, no more than about 1 μm, no more than about 0.1 μm, no more than about 0.01 μm, no more than about 0.001 μm, or no more than about 0.0001 μm.

Combinations of the above-referenced distance between chambers are also possible (e.g., at least about 0.0001 μm and no more than about 2,000 μm or at least about 1 μm and no more than about 20 μm) inclusive of all values and ranges therebetween.

In some embodiments, a surface can couple with the chamber C to cover the chamber C. In some embodiments of systems with multiple chambers, the surface can fluidically isolate chambers from one another. For example, the surface can isolate a first chamber from a second chamber. In some embodiments, the surface can include glass.

As shown in FIG. 2A through FIG. 3B, chambers of the multiplex assay chip device can include one or more pockets within at least one first region. As in FIG. 2A, the chamber includes two pockets (P(1) and P(2)) within the at least one first region. In some embodiments, each chamber in the multiplex assay chip device can include, within the at least one first region, at least about 2 pockets, at least about 3 pockets, at least about 4 pockets, at least about 5 pockets, at least about 6 pockets, at least about 7 pockets, at least about 8 pockets, at least about 9 pockets, or at least about 10 pockets. In some embodiments, each chamber in the multiplex assay chip device can include, within the at least one first region, no more than about 10 pockets, no more than about 9 pockets, no more than about 8 pockets, no more than about 7 pockets, no more than about 6 pockets, no more than about 5 pockets, no more than about 4 pockets, no more than about 3 pockets, or no more than about 2 pockets.

Combinations of the above-referenced number of pockets within the at least one first region of each chamber in the multiplex assay chip device are also possible (e.g., at least about 2 pockets and no more than about 10 pockets or at least about 2 pockets and no more than about 4 pockets), inclusive of all values and ranges therebetween. In some embodiments, the at least one first region of each chamber in the multiplex assay chip device can include about 2 pockets, about 3 pockets, about 4 pockets, about 5 pockets, about 6 pockets, about 7 pockets, about 8 pockets, about 9 pockets, about 10 pockets.

In some embodiments, the at least one first region, and the pockets therein, can be composed of glass, polymers, metal, silica, or any other suitable material. In some embodiments, the pockets can have a circular cross section, a rectangular cross section, a square cross section, a pentagonal cross section, an elliptical cross section, a hexagonal cross section, or any other suitable cross-sectional shape.

In some embodiments, the at least one first region has a circular cross section having a diameter or cross-sectional width of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, or at least about 900 μm. In some embodiments, the at least one first region having a circular cross section can have a diameter or cross-sectional width of no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced diameters or cross-sectional widths of the circular at least one first region is also possible (e.g., at least about 1 μm and no more than about 1,000 μm, at least about 5 μm and no more than about 200 μm, or at least about 5 μm and no more than about 20 μm) inclusive of all values and ranges therebetween. In some embodiments, the circular at least one first region can have a diameter or cross-sectional width of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, or about 1,000 μm.

In some embodiments, wherein the at least one first region includes at least two first regions having circular cross sections, each of the at least two first regions can have uniform diameters or cross-sectional widths. In some embodiments, wherein the at least one first region includes at least two first regions having circular cross sections, each of the at least two first regions can have different diameters or cross-sectional widths.

In some embodiments, wherein the at least one first region is two or more first regions, a difference in diameter between first regions of the multiplex assay chip device can be at least about 5 μm, at least about 10 μm, at least about 15 μm, at least about 20 μm, at least about 25 μm, at least about 30 μm, at least about 35 μm, at least about 40 μm, or at least about 45 μm. In some embodiments, the difference in diameter between first regions of the multiplex assay chip device can be no more than about 50 μm, no more than about 45 μm, no more than about 40 μm, no more than about 35 μm, no more than about 30 μm, no more than about 25 μm, no more than about 20 μm, no more than about 15 μm, or no more than about 10 μm. Combinations of the above-referenced differences in diameter between the first regions of the multiplex assay chip device are also possible (e.g., at least about 5 μm and no more than about 50 μm or at least about 20 μm and no more than about 40 μm), inclusive of all values and ranges therebetween. In some embodiments, the difference in diameter between first regions of the multiplex assay chip device can be about 5 μm, about 10 μm, about 15 μm, about 20 μm, about 25 μm, about 30 μm, about 35 μm, about 40 μm, about 45 μm, or about 50 μm. In some embodiments, one or more of the first regions can be center aligned with respect to the width of an adjacent chamber. In some embodiments, one or more of the first regions can be not center aligned with respect to the width of an adjacent chamber. In some embodiments, the first regions can be non-overlapping, positioned at an edge, or positioned elsewhere.

In some embodiments, the multiplex assay chip device can include at least about 1,000 chambers, at least about 2,000 chambers, at least about 2,500 chambers, at least about 3,000 chambers, at least about 4,000 chambers, at least about 5,000 chambers, at least about 6,000 chambers, at least about 7,000 chambers, at least about 8,000 chambers, at least about 9,000 chambers, at least about 10,000 chambers, at least about 11,000 chambers, at least about 12,000 chambers, at least about 13,000 chambers, at least about 14,000 chambers, at least about 15,000 chambers, at least about 16,000 chambers, at least about 17,000 chambers, at least about 18,000 chambers, at least about 19,000 chambers, at least about 20,000 chambers, at least about 50,000 chambers, at least about 100,000 chambers, or at least about 200,000 chambers. In some embodiments, the multiplex assay chip device can include no more than about 200,000 chambers, no more than about 100,000 chambers, no more than about 50,000 chambers, no more than about 20,000 chambers, no more than about 19,000 chambers, no more than about 18,000 chambers, no more than about 17,000 chambers, no more than about 16,000 chambers, no more than about 15,000 chambers, no more than about 14,000 chambers, no more than about 13,000 chambers, no more than about 12,000 chambers, no more than about 11,000 chambers, no more than about 10,000 chambers, no more than about 9,000 chambers, no more than about 8,000 chambers, no more than about 7,000 chambers, no more than about 6,000 chambers, no more than about 5,000 chambers, no more than about 4,000 chambers, no more than about 3,000 chambers, no more than about 2,500 chambers, no more than about 2,000 chambers, or no more than about 1,000 chambers.

Combinations of the above-referenced number of chambers in the multiplex assay chip device are also possible (e.g., at least about 20,000 chambers and no more than about 200,000 chambers or at least about 10,000 chambers and no more than about 100,000 chambers), inclusive of all values and ranges therebetween. In some embodiments, the multiplex assay chip device can include about 1,000 chambers, about 2,000 chambers, about 2,500 chambers, about 3,000 chambers, about 4,000 chambers, about 5,000 chambers, about 6,000 chambers, about 7,000 chambers, about 8,000 chambers, about 9,000 chambers, about 10,000 chambers, about 11,000 chambers, about 12,000 chambers, about 13,000 chambers, about 14,000 chambers, about 15,000 chambers, about 16,000 chambers, about 17,000 chambers, about 18,000 chambers, about 19,000 chambers, about 20,000 chambers, about 50,000 chambers, about 100,000 chambers, or about 200,000 chambers.

In some embodiments, the chambers can be arranged in an array of m chambers by n chambers, wherein m and n are both positive integers. In some embodiments, m can be equal to n. In some embodiments, m can be greater than n or less than n.

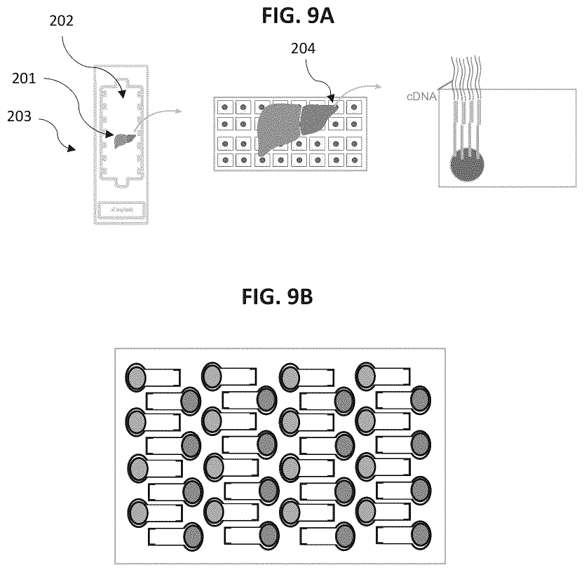

As shown in FIGS. 9A and 9B, the at least one first region of each chamber may include a capture bead disposed therein.

In some embodiments, m can be at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, or at least about 1,000. In some embodiments, m can be no more than about 1,000, no more than about 900, no more than about 800, no more than about 700, no more than about 600, no more than about 500, no more than about 400, no more than about 300, no more than about 250, no more than about 200, no more than about 190, no more than about 180, no more than about 170, no more than about 160, no more than about 150, no more than about 140, no more than about 130, no more than about 120, no more than about 110, or no more than about 100. Combinations of the above-referenced values for m are also possible (e.g., at least about 100 and no more than about 1,000, or at least about 300 and no more than about 600), inclusive of all values and ranges therebetween. In some embodiments, m can be about 100, about 110, about 120, about 130, about 140, about 150, about 160, about 170, about 180, about 190, about 200, about 250, about 300, about 350, about 400, about 450, about 500, about 600, about 700, about 800, about 900, or about 1,000.

In some embodiments, n can be at least about 100, at least about 110, at least about 120, at least about 130, at least about 140, at least about 150, at least about 160, at least about 170, at least about 180, or at least about 190, at least about 200, at least about 250, at least about 300, at least about 350, at least about 400, at least about 450, at least about 500, at least about 600, at least about 700, at least about 800, at least about 900, or at least about 1,000. In some embodiments, n can be no more than about 1,000, no more than about 900, no more than about 800, no more than about 700, no more than about 600, no more than about 500, no more than about 400, no more than about 300, no more than about 250, no more than about 200, no more than about 190, no more than about 180, no more than about 170, no more than about 160, no more than about 150, no more than about 140, no more than about 130, no more than about 120, no more than about 110, or no more than about 100. Combinations of the above-referenced values for n are also possible (e.g., at least about 100 and no more than about 1,000, or at least about 300 and no more than about 600), inclusive of all values and ranges therebetween. In some embodiments, n can be about 100, about 110, about 120, about 130, about 140, about 150, about 160, about 170, about 180, about 190, about 200, about 250, about 300, about 350, about 400, about 450, about 500, about 600, about 700, about 800, about 900, or about 1,000.

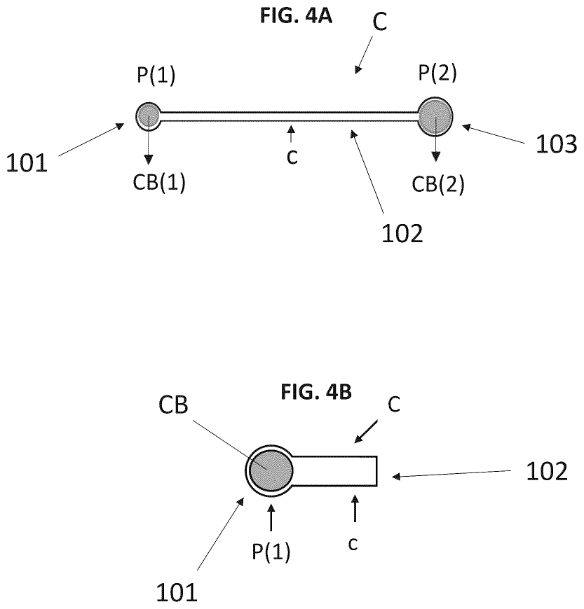

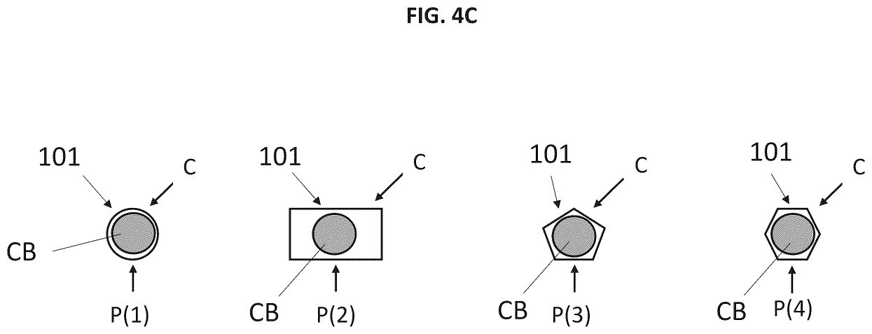

Returning now to FIG. 4A through FIG. 4C, schematic depictions of shapes are provided, according to embodiments. The multiplex assay chip devices can be used for multiplexed analysis of biological material. The multiplex assay chip devices include a plurality of chambers such as chamber C having at least one first region and, optionally, at least one second region. Moreover, the multiplex assay chip devices include, within the at least one first region, pocket(s) (e.g., pockets P(1) and P(2) of FIG. 4A) with capture bead(s) (e.g., capture beads CB(1) and CB(2) of FIG. 4A) disposed therein.

In some embodiments, the multiplex assay chip device can include at least about 1,000 capture beads, at least about 2,000 capture beads, at least about 2,500 capture beads, at least about 3,000 capture beads, at least about 4,000 capture beads, at least about 5,000 capture beads, at least about 6,000 capture beads, at least about 7,000 capture beads, at least about 8,000 capture beads, at least about 9,000 capture beads, at least about 10,000 capture beads, at least about 11,000 capture beads, at least about 12,000 capture beads, at least about 13,000 capture beads, at least about 14,000 capture beads, at least about 15,000 capture beads, at least about 16,000 capture beads, at least about 17,000 capture beads, at least about 18,000 capture beads, at least about 19,000 capture beads, at least about 20,000 capture beads, at least about 30,000 capture beads, at least about 40,000 capture beads, at least about 50,000 capture beads, at least about 60,000 capture beads, at least about 70,000 capture beads, at least about 80,000 capture beads, at least about 90,000 capture beads, at least about 100,000 capture beads, or at least about 200,000 capture beads. In some embodiments, the multiplex assay chip device can include no more than about 200,000 capture beads, no more than about 100,000 capture beads, no more than about 90,000 capture beads, no more than about 80,000 capture beads, no more than about 70,000 capture beads, no more than about 60,000 capture beads, no more than about 50,000 capture beads, no more than about 40,000 capture beads, no more than about 30,000 capture beads, no more than about 20,000 capture beads, no more than about 15,000 capture beads, no more than about 14,000 capture beads, no more than about 13,000 capture beads, no more than about 12,000 capture beads, no more than about 11,000 capture beads, no more than about 10,000 capture beads, no more than about 9,000 capture beads, no more than about 8,000 capture beads, no more than about 7,000 capture beads, no more than about 6,000 capture beads, no more than about 5,000 capture beads, no more than about 4,000 capture beads, no more than about 3,000 capture beads, no more than about 2,500 capture beads, no more than about 2,000 capture beads, or no more than about 1,000 capture beads.

Combinations of the above-referenced number of capture beads in the multiplex assay chip device are also possible (e.g., at least about 10,000 capture beads and no more than about 40,000 capture beads or at least about 20,000 capture beads and no more than about 100,000 capture beads), inclusive of all values and ranges therebetween. In some embodiments, the multiplex assay chip device can include about 1,000 capture beads, about 2,000 capture beads, about 2,500 capture beads, about 3,000 capture beads, about 4,000 capture beads, about 5,000 capture beads, about 6,000 capture beads, about 7,000 capture beads, about 8,000 capture beads, about 9,000 capture beads, about 10,000 capture beads, about 11,000 capture beads, about 12,000 capture beads, about 13,000 capture beads, about 14,000 capture beads, about 15,000 capture beads, about 16,000 capture beads, about 17,000 capture beads, about 18,000 capture beads, about 19,000 capture beads, about 20,000 capture beads, about 30,000 capture beads, about 40,000 capture beads, about 50,000 capture beads, about 60,000 capture beads, about 70,000 capture beads, about 80,000 capture beads, about 90,000 capture beads, about 100,000 capture beads, or about 200,000 capture beads .

In some embodiments, the capture beads can have a diameter or cross-sectional width of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, or at least about 100 μm. In some embodiments, the capture beads can have a diameter or cross-sectional width of no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced diameters or cross-sectional widths of the capture beads are also possible (e.g., at least about 1 μm and no more than about 20 μm, at least about 5 μm and no more than about 50 μm, or at least about 5 μm and no more than about 100 μm) inclusive of all values and ranges therebetween. In some embodiments, the capture beads can have a diameter or cross-sectional width of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, or about 100 inn.

In some embodiments, the capture beads can have smaller diameters or cross-sectional widths than a corresponding pocket of at least one first region, in which the capture beads are disposed. For example, as in FIG. 4A, the capture bead CB(1) can have a smaller diameter than the pocket (P1). In some embodiments, a capture bead can have a smaller diameter, cross-sectional width, and/or cross-sectional length than a pocket, in which the capture bead is disposed by at least about 5 μm, at least about 10 μm, at least about 15 μm, at least about 20 μm, at least about 25 μm, at least about 30 μm, at least about 35 μm, at least about 40 μm, and/or at least about 45 μm. In some embodiments, a capture bead can have a smaller diameter, cross-sectional width, and/or cross-section length than a pocket of at least one first region, in which the capture bead is disposed by no more than about 50 μm, no more than about 45 μm, no more than about 40 μm, no more than about 35 μm, no more than about 30 μm, no more than about 25 μm, no more than about 20 μm, no more than about 15 μm, or no more than about 10 μm. Combinations of the above-referenced differences between the diameters or cross-sectional dimensions of a pocket of at least one first region and a capture bead disposed in the pocket of the at least one first region are also possible (e.g., at least about 5 μm, and no more than about 50 μm or at least about 20 μm and no more than about 40 μm), inclusive of all values and ranges therebetween. In some embodiments, a capture bead can have a smaller diameter or cross-sectional dimension than a pocket of at least one first region, in which the capture bead is disposed by about 5 μm, about 10 μm, about 15 μm, about 20 μm, about 25 μm, about 30 μm, about 35 μm, about 40 μm, about 45 μm, or about 50 inn.

In some embodiments, the capture beads within at least first region can have larger diameters or cross-sectional dimensions than adjacent regions of the chamber. For example, the capture bead CB(1) can have a larger diameter than at least one dimension of channel c, such that the capture bead will not fit within channel c. In some embodiments, a capture bead can have a larger diameter or cross-sectional dimension than adjacent regions of the chamber by at least about 5 μm, at least about 10 μm, at least about 15 μm, at least about 20 μm, at least about 25 μm, at least about 30 μm, at least about 35 μm, at least about 40 μm, at least about 45 μm. In some embodiments, a capture bead can have a larger diameter or cross-sectional dimension than adjacent regions of the chamber by no more than about 50 μm, no more than about 45 μm, no more than about 40 μm, no more than about 35 μm, no more than about 30 μm, no more than about 25 μm, no more than about 20 μm, no more than about 15 μm, or no more than about 10 μm. Combinations of the above-referenced differences between the diameters or cross-sectional dimensions of a capture bead and adjacent regions of the chamber are also possible (e.g., at least about 5 μm, and no more than about 50 μm or at least about 20 μm and no more than about 40 μm), inclusive of all values and ranges therebetween. In some embodiments, a capture bead can have a larger diameter or cross-sectional dimension than adjacent regions of the chamber by about 5 μm, about 10 μm, about 15 μm, about 20 μm, about 25 μm, about 30 μm, about 35 μm, about 40 μm, about 45 μm, or about 50 μm.

In some embodiments, the capture beads can all have substantially the same size. In some embodiments, the capture beads can be of different sizes. For example, as shown in FIG. 4A capture bead CB(2) can be larger than capture bead CB(1), such that the capture bead CB(2) cannot fit in the pocket P(1). As an additional example capture CB(2) can have a diameter or cross-sectional dimension smaller than a diameter or cross-sectional width of pocket P(2), yet larger than a diameter or cross sectional dimension of pocket P(1), such that capture bead CB(2) can only fit into pocket P(2).

In some embodiments, the capture beads (e.g., CB(1), CB(2)) can include a capture moiety. In some embodiments, the capture moiety can capture nucleic acid sequences, peptides, proteins, metabolites, original molecules, or any combination thereof. In some embodiments, the capture moiety can capture DNA, RNA, or a combination thereof. In some embodiments, the DNA can include autosomal DNA, chromosomal DNA, cDNA, exosome DNA, single stranded DNA, double stranded DNA, or any combination thereof. In some embodiments, the RNA can include mRNA, rRNA, tRNA, snRNA, regulatory RNA, double stranded RNA, microRNA, exosome RNA, or any combination thereof. In some embodiments, the RNA can include a guide RNA from a CRISPR-Cas system. In some embodiments, the capture beads can include an oligonucleotide capture bead comprising a nucleic acid capture sequence tethered to a bead.

In some embodiments, the capture bead can include at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900 , at least about 1,000, at least about 10,000, at least about 100,000, at least about 1,000,000, or at least about 10,000,000 capture moieties. In some embodiments, the capture bead can include no more than about 10,000,000, no more than about 1,000,000, no more than about 100,000, no more than about 10,000, no more than about 1,000, no more than about 900, no more than about 800, no more than about 700, no more than about 600, no more than about 500, no more than about 400, no more than about 300, no more than about 200, no more than about 100, no more than about 90, no more than about 80, no more than about 70, no more than about 60, no more than about 50, no more than about 40, no more than about 30, no more than about 20, no more than about 10, no more than about 9, no more than about 8, no more than about 7, no more than about 6, no more than about 5, no more than about 4, no more than about 3, or no more than about 2 capture moieties.

Combinations of the above-referenced numbers of capture moieties are also possible (e.g., at least about 1 and no more than about 10,000,000 capture moieties or at least about 100 and no more than about 500 capture moieties), inclusive of all values and ranges therebetween. In some embodiments, the capture bead can include about 1, about 2, about 3, about 4, about 5, about 6, about 7, about 8, about 9, about 10, about 20, about 30, about 40, about 50, about 60, about 70, about 80, about 90, about 100, about 200, about 300, about 400, about 500, about 600, about 700, about 800, about 900, about 1,000, about 10,000, about 100,000, about 1,000,000, or about 10,000,000 capture moieties.

In some embodiments, the nucleic acid capturing bead can include at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900 , at least about 1,000, at least about 10,000, at least about 100,000, at least about 1,000,000, or at least about 10,000,000 capture nucleic acid sequences. In some embodiments, the nucleic acid capturing bead can include no more than about 10,000,000 no more than about 1,000,000, no more than about 100,000, no more than about 10 no more than about 1,000, no more than about 900, no more than about 800, no more than about 700, no more than about 600, no more than about 500, no more than about 400, no more than about 300, no more than about 200, no more than about 100, no more than about 90, no more than about 80, no more than about 70, no more than about 60, no more than about 50, no more than about 40, no more than about 30, no more than about 20, no more than about 10, no more than about 9, no more than about 8, no more than about 7, no more than about 6, no more than about 5, no more than about 4, no more than about 3, or no more than about 2 capture nucleic acid sequences.

Combinations of the above-referenced numbers of capture nucleic acid sequences are also possible (e.g., at least about 1 and no more than about 10,000,000 capture nucleic acid sequences or at least about 100 and no more than about 500 capture nucleic acid sequences), inclusive of all values and ranges therebetween. In some embodiments, the nucleic acid capturing bead can include about 1, about 2, about 3, about 4, about 5, about 6, about 7, about 8, about 9, about 10, about 20, about 30, about 40, about 50, about 60, about 70, about 80, about 90, about 100, about 200, about 300, about 400, about 500, about 600, about 700, about 800, about 900, about 1,000, about 10,000, about 100,000, about 1,000,000, or about 10,000,000 capture nucleic acid sequences.

In some embodiments, the capture nucleic acid sequence can include an individually unique chamber barcode sequence, a PCR handle, a unique molecular identifier (UMI), a barcode handle sequence, and a capture sequence. In some embodiments, the capture nucleic acid sequence can include an individually unique chamber barcode sequence, a PCR handle, a unique molecular identifier (UMI), a template switch oligonucleotide (TSO) sequence, a barcode handle sequence, and a capture sequence. In some embodiments, the capture sequence can include a polyT sequence for mRNA polyA capture. In some embodiments, the capture sequence can include an rGrGrG capture sequence for mRNA capture, wherein rGrGrG denotes three riboguanosines. In some embodiments, the capture sequence can include a gene-specific or sequence-specific capture sequence. In some embodiments, each nucleic acid sequence of the nucleic acid capturing bead can include a unique UMI. In some embodiments, the barcode sequence of the capture bead can be unique to each capture bead. In some embodiments, the barcode sequence of the capture bead can be unique to each chamber. In some embodiments, the capture moiety can capture proteins. In some embodiments, the capture beads can include a capture antibody tethered to a bead. In some embodiments, the protein capturing CB comprises an antibody, an aptamer, a functional fragment of an antibody, or an antibody mimetic. In some embodiments, the capture beads can include plastic, polymer, metal, silica, or any other suitable material, or combinations thereof.

In some embodiments, the capture beads can include a porous capture bead. In some embodiments, the porous capture bead can release one or more agents. In some embodiments, the one or more agents can include an enzyme, a catalyzer, a stimulatory agent, a therapeutic agent, or any combination thereof.

In some embodiments, the biological material can include a biological sample, a metabolite, a protein, a polypeptide, a cell, or any combination thereof. In some embodiments, the biological sample is a sectioned histological sample.

In some embodiments, the cell can be a single cell. In some embodiments, the single cell can include a healthy cell. In some embodiments, the single cell can include a tumor cell. In some embodiments, the healthy cell can include a malignant cell. In some embodiments, the single cell can include a neural cell. In some embodiments, the single cell can include a glial cell. In some embodiments, the single cell can include an immune cell. In some embodiments, the single cell can include a T-cell and/or a B-cell. In some embodiments, the single cell can include a bacterium. In some embodiments, the single cell can include a Chinese hamster ovary (CHO) cell. In some embodiments, the single cell can include a yeast cell. In some embodiments, the single cell can include an alga.

In some embodiments, the single cell can be genetically modified. In some embodiments, the biological sample can be obtained from a subject. In some embodiments, the biological sample can include blood, cerebral spinal fluid (CSF) lymph fluid, plural effusion, urine, saliva, or any combination thereof. In some embodiments, the biological sample can include a cell culture media. In some embodiments, the biological sample can include a tissue sample or tissue biopsy. In some embodiments, the subject can be healthy. In some embodiments, the subject can have cancer. In some embodiments, the subject can have an infection. In some embodiments, the subject can have an autoimmune disorder. In some embodiments, the subject can have an inflammatory disorder. In some embodiments, the subject can have a neurological disorder. In some embodiments, the subject can have a metabolic disorder. In some embodiments, the subject can have a degenerative disorder. In some embodiments, the subject can have a genetic mutation or epigenetic modification associated with a disease or disorder.

In embodiments, for schematics including those of FIG. 2A through FIG. 3B, the channel c (of the at least one second region) of the chamber C may include capture antibodies therein. In embodiments, the antibodies may be printed directly into one or more of the at least one first region and the at least one second region of the substrate forming the chamber C. Additionally, or alternatively, the antibodies may be printed onto a glass substrate, polymer substrate, or another substate meant to overlap with the at least one first region and/or at least one second region of the chamber C when the surface is exposed to the chamber C.

In embodiments, the capture antibodies may be printed directly onto the at least one second region of the substrate forming the chamber C. In other embodiments, the antibodies may be printed directly onto the surface that is exposed to the chamber C. In each embodiment, or in a combination thereof, the antibodies printed thereon are spatially arranged to overlap with a specific position of the chamber C to allow for co-location of relevant transcriptomic, proteomic, and genomic results.

To this end, the surface can couple with the chamber C to cover the chamber C. In some embodiments of systems with multiple chambers, the surface can fluidically isolate chambers from one another. For example, the surface can isolate a first chamber from a second chamber. In some embodiments, the surface can include glass.

In some embodiments, wherein the antibodies are printed directly onto the surface of the glass which can couple with the chamber C to cover the chamber C, the surface can include a plurality of substantially parallel lines of capture antibodies. In some embodiments, each line of capture antibodies can include a different antibody, relative to antibodies of adjacent lines of capture antibodies. In some embodiments, each antibody can be configured to bind to a different target molecule. In some embodiments, at least a portion of each of the plurality of substantially parallel lines are arranged to be exposed to each chamber.

In some embodiments, the plurality of substantially parallel lines of capture antibodies can include at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, or at least about 190 different capture antibodies. In some embodiments, the plurality of substantially parallel lines of capture antibodies can include no more than about 200, no more than about 190, no more than about 180, no more than about 170, no more than about 160, no more than about 150, no more than about 140, no more than about 130, no more than about 120, no more than about 110, no more than about 100, no more than about 90, no more than about 80, no more than about 70, no more than about 60, no more than about 50, no more than about 40, no more than about 30, no more than about 20, no more than about 10, no more than about 9, no more than about 8, no more than about 7, no more than about 6, no more than about 5, no more than about 4, or no more than about 3 different capture antibodies. Combinations of the above-referenced numbers of different capture antibodies are also possible (e.g., at least about 2 and no more than about 200 or at least about 2 and no more than about 50), inclusive of all values and ranges therebetween. In some embodiments, the plurality of substantially parallel lines of capture antibodies can include about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 40, about 50, about 60, about 70, about 80, about 90, about 100, about 110, about 120, about 130, about 140, about 150, about 160, about 170, about 180, about 190, or about 200 different capture antibodies. In some embodiments, each chamber of the plurality of chambers is configured to contact the plurality of plurality of substantially parallel lines of capture antibodies at least once. In some embodiments, each chamber of the plurality of chambers is configured to contact the plurality of plurality of substantially parallel lines of capture antibodies at least twice (i.e. each capture antibody of the plurality of substantially parallel lines of capture antibodies contacts the chamber at least twice).

In some embodiments, each substantially parallel line of capture antibodies comprises a single species of antibody specific for a single biological target molecule. In some embodiments, each substantially parallel line of capture antibodies can comprise at least two species of antibody, each species specific for a different biological target molecule. In some embodiments, each substantially parallel line of capture antibodies can comprise at least three species of antibody, each species specific for a different biological target molecule. In some embodiments, each substantially parallel line of capture antibodies can comprise at least four species of antibody, each species specific for a different biological target molecule. In cases where a substantially parallel line of capture antibodies comprises two or more species of capture antibodies, each species of capture antibody can be detected by a distinct secondary detection antibody each having a spectrally distinguishable label (i.e., fluorescent label).

In some embodiments, wherein the antibodies are printed directly into one or more of the at least one first region and the at least one second region of the substrate forming the chamber C, the antibodies may be printed onto at least one surface of the one or more of the at least one first region and the at least one second region of the substrate. In embodiments, the printed antibodies include a plurality of substantially parallel lines of capture antibodies. In some embodiments, each line of capture antibodies can include a different antibody, relative to antibodies of adjacent lines of capture antibodies. In some embodiments, each antibody can be configured to bind to a different target molecule. In some embodiments, at least a portion of each of the plurality of substantially parallel lines are arranged to be exposed to each chamber.

In some embodiments, the plurality of substantially parallel lines of capture antibodies can include at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, or at least about 190 different capture antibodies. In some embodiments, the plurality of substantially parallel lines of capture antibodies can include no more than about 200, no more than about 190, no more than about 180, no more than about 170, no more than about 160, no more than about 150, no more than about 140, no more than about 130, no more than about 120, no more than about 110, no more than about 100, no more than about 90, no more than about 80, no more than about 70, no more than about 60, no more than about 50, no more than about 40, no more than about 30, no more than about 20, no more than about 10, no more than about 9, no more than about 8, no more than about 7, no more than about 6, no more than about 5, no more than about 4, or no more than about 3 different capture antibodies. Combinations of the above-referenced numbers of different capture antibodies are also possible (e.g., at least about 2 and no more than about 200 or at least about 2 and no more than about 50), inclusive of all values and ranges therebetween. In some embodiments, the plurality of substantially parallel lines of capture antibodies can include about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 40, about 50, about 60, about 70, about 80, about 90, about 100, about 110, about 120, about 130, about 140, about 150, about 160, about 170, about 180, about 190, or about 200 different capture antibodies. In some embodiments, each chamber of the plurality of chambers is configured to contact the plurality of plurality of substantially parallel lines of capture antibodies at least once. In some embodiments, each chamber of the plurality of chambers is configured to contact the plurality of plurality of substantially parallel lines of capture antibodies at least twice (i.e. each capture antibody of the plurality of substantially parallel lines of capture antibodies contacts the chamber at least twice).

In some embodiments, each substantially parallel line of capture antibodies comprises a single species of antibody specific for a single biological target molecule. In some embodiments, each substantially parallel line of capture antibodies can comprise at least two species of antibody, each species specific for a different biological target molecule. In some embodiments, each substantially parallel line of capture antibodies can comprise at least three species of antibody, each species specific for a different biological target molecule. In some embodiments, each substantially parallel line of capture antibodies can comprise at least four species of antibody, each species specific for a different biological target molecule. In cases where a substantially parallel line of capture antibodies comprises two or more species of capture antibodies, each species of capture antibody can be detected by a distinct secondary detection antibody each having a spectrally distinguishable label (i.e., fluorescent label).

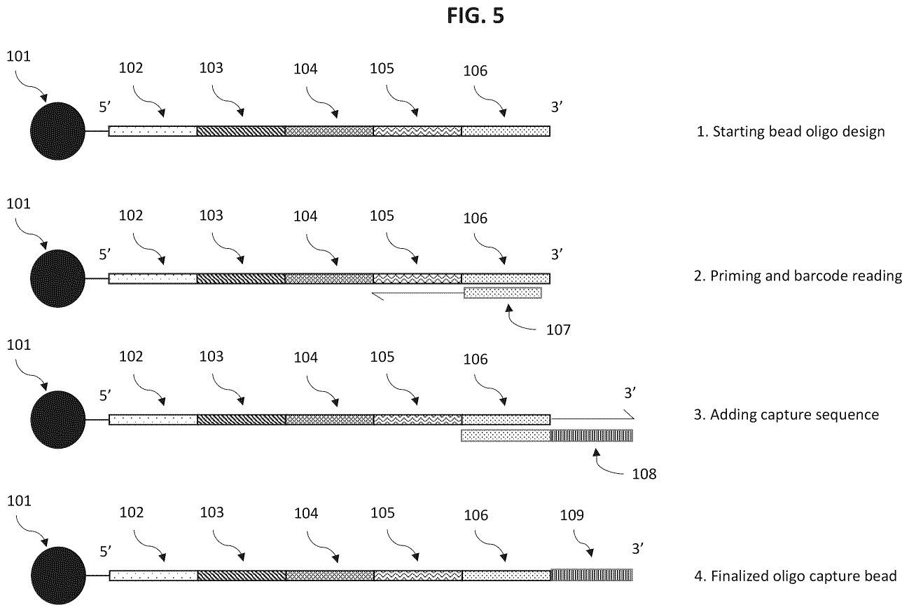

With reference now to FIG. 5, a schematic detailing the creation of a nucleic acid capture bead is disclosed.

First, a bead 101 comprising at least one nucleic acid sequence comprising from 5′ to 3′ a 5T spacer sequence 102, a PCR handle sequence 103, a unique molecular identifier (UMI) sequence 104, chamber barcode sequence 105, and sequencing (seq) handle 106 is obtained. Next, the chamber barcode sequence 105 can be read by hybridizing a barcode reading primer 107 to the seq handle sequence 106 and extending the primer using the chamber barcode sequence as a template. Sequencing the chamber barcode can be performed “on chip” or on substrate using fluorescently labeled nucleotides and a fluorescent imaging system. In some embodiments, a capture sequence can be added to the capture bead nucleic acid sequence by hybridizing an oligonucleotide sequence comprising a capture sequence template 108 to the sequence handle 106 and extending the sequence using PCR to produce, as in the bottom image, a bead 101 comprising at least one nucleic acid sequence comprising from 5′ to 3′ a 5T spacer sequence 102, a PCR handle sequence 103, a UMI sequence 104, chamber barcode sequence 105 chamber barcode(individually unique chamber barcode), a seq handle 106, and a capture sequence 109.

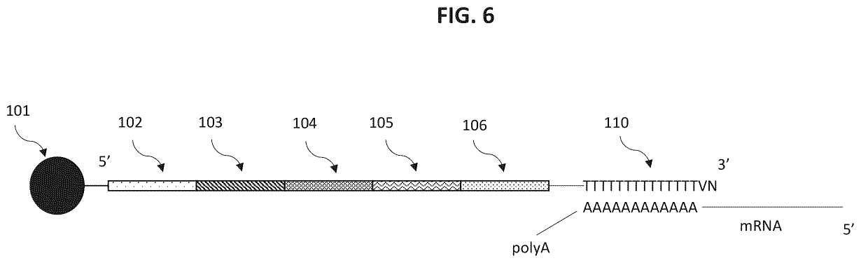

FIG. 6 is an image of a capture bead comprising from 5′ to 3′ a 5T spacer sequence 102, a PCR handle sequence 103, a UMI sequence 104, chamber barcode sequence 105 (individually unique chamber barcode), a seq handle 106, and configured to capture mRNA sequences by hybridizing a polyT capture sequence 110 to the 3′ polyA sequence of an mRNA.

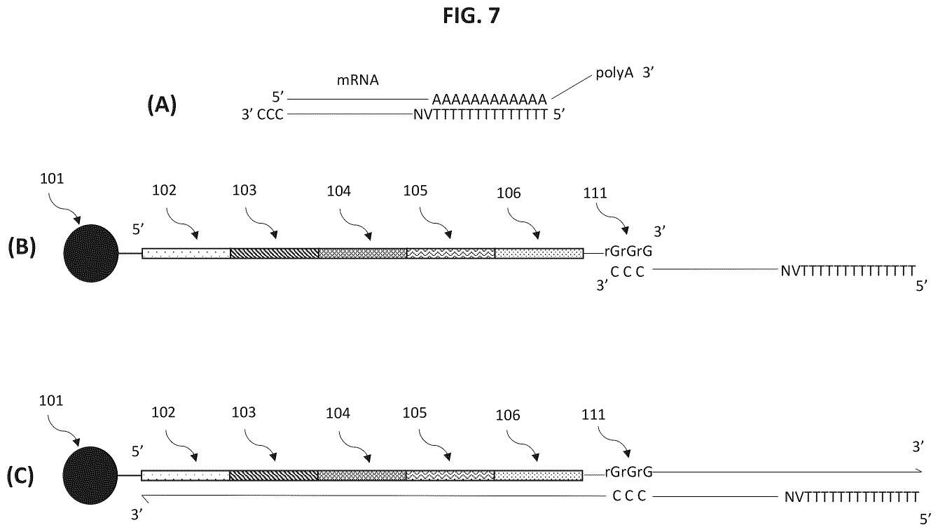

FIG. 7 is a schematic detailing 5′ mRNA capture using capture beads of the disclosure configured to comprise rGrGrG (three riboguanosines) for capture of 5′ mRNA sequences that have been reverse transcribed to include a terminal polyC sequence. In (A), mRNA is reverse transcribed using an oligo-dTVN primer and a reverse transcriptase having terminal transferase activity, which, upon reaching the 5′ end of the RNA template, adds a few additional nucleotides (mostly deoxycytidine) to the 3′ end of the newly synthesized cDNA strand. In (B), the 3′ dC extension of the cDNA molecule generated in (A) hybridizes to the rGrGrG sequences at the 3′ end of the nucleic acid sequence comprising from 5′ to 3′ a 5T spacer sequence 102, a PCR handle sequence 103, a UMI sequence 104, chamber barcode sequence 105, a seq handle 106, and a rGrGrG sequence 111. Extension of the 3′ ends of both molecules shown in (B) results in the double-stranded nucleic acid shown in (C).

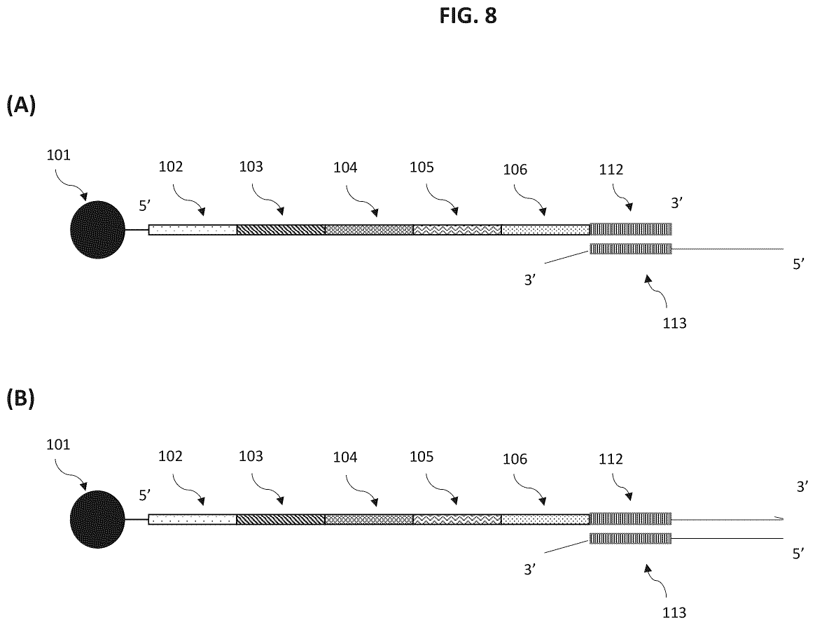

FIG. 8 is an image of a capture bead configured to capture nucleic sequences by hybridizing a sequence specific capture sequence 112 to a portion of a target nucleic acid 113, such as an RNA or DNA sequence. In some embodiments, this is a gene-specific capture sequence. In (A), target nucleic acid 113 hybridizes to the sequence specific capture sequence 112 at the 3′ end of the nucleic acid sequence comprising from 5′ to 3′ a 5T spacer sequence 102, a PCR handle sequence 103, a UMI sequence 104, chamber barcode sequence 105, a seq handle 106, and sequence specific capture sequence 112. The 3′ end of the sequence specific capture sequence 112 is extended using target nucleic acid 113 as the template as shown in (B).

In some embodiments, devices of the present disclosure, as well as structure and components thereof, can be combined with or modified from devices and structure and components thereof disclosed in any of U.S. Pat. Nos. 10,584,366 and 9,506,917, as well as U.S. Publication Nos. 2017-0138942, 2020-0166518, 2019-0285626, and 2019-0376898. The contents of each of the foregoing are incorporated by reference herein in its entirety.

### Spatially Resolved Analysis of Biological Molecules

The disclosure provides methods of performing spatially resolved analysis of biological material in biological samples. The distribution of biological material, such as biomolecules including proteins, metabolites, and nucleic acids, across an organism is highly heterogeneous and variable over time. This heterogeneity is not limited to changes in distribution between organs or cell type. In fact, changes in the distribution of biomolecules can be observed within small subsections of organs or across sections of a tumor. This distribution can also be observed from one single cell to another single cell, even in single cells directly adjoining one another in a tissue, organ, or tumor. Methods capable of detecting this heterogeneity of expression in a spatially resolved fashion can provide critical insight into the biology of an organism including the development or progression of diseases, such as neurodegenerative diseases or cancer. Spatially resolved methods of analyzing biological molecules in a biological sample enable increased understanding of development and progression of disease, thereby offering the potential development of new therapeutic strategies for combatting disease.

The disclosure provides a method for determining spatially resolved information from a biological sample, comprising: I) providing a device comprising a substrate having a plurality of chambers each comprising at least one first capture bead (CB); II) obtaining a sectioned histological sample from a subject mounted to a surface; III) treating the sectioned histological sample with a cell lysis or permeabilization reagent, under conditions sufficient for target biological molecules to be released from the sample; IV) coupling the surface to the substrate such that the surface seals the plurality of chambers forming a plurality of enclosures; wherein each enclosure comprises a first CB in fluid communication with a portion of the sectioned histological sample and target biological molecules; V) incubating the sample under conditions sufficient for the target biological molecules to contact the CB to form CB-target biological molecule complexes; and VI) detecting the complexed target biological molecules.

Methods and devices of the disclosure enable the biological molecules captured by capture beads present in each chamber of the plurality of chambers of the substrate to be linked back to the exact location and chamber on the array of chambers. In other words, each chamber serves as a pixel that can record the biological molecules released by the sample at that location. Methods of the disclosure provide for methods of detecting, identifying, or sequencing the biological material detected by the bead at each location. Further, devices of the disclosure are configured such that the spatial location of each biological molecule is traceable and stored. As such, following detecting, identifying, or sequencing the biological material for the entire sample, the data belonging to each pixel (i.e. the biological material captured by each capture bead within a specific chamber) can be processed and a spatial distribution of the biological material can be generated reflecting the relative levels of each biomolecule at each discrete location of the biological sample.

FIG. 9A is a schematic detailing a device and method for determining spatially resolved information from a sectioned histological sample. Histological sample 201 is mounted to a slide 202 of a chip device 203 comprising a plurality of chambers (see, e.g., chamber 204), each comprising a capture bead configured to capture nucleic acid sequences such as mRNA. The chambers shown in FIG. 9A are generally square, but the chambers can have other shapes such as those shown in FIG. 4. Each chamber and capture bead residing in the chamber contact a discrete section of the sample and capture the nucleic acids within that section of the sample, which provides spatial resolution as to the location in the histological sample from which the nucleic acids were captured. Captured nucleic acid sequences can then be converted to cDNA and sequenced using downstream sequencing technologies. Capture beads each have a unique predetermined chamber barcode sequence that is incorporated into the synthesized cDNA sequences making it possible to track each sequence back to the chamber in which it was captured. Prior to the use of the device for the analysis of a histological sample, the chamber barcode sequence of the capture bead oligonucleotides is determined by “on chip,” or in chamber, sequencing.

In embodiments, and with reference to the above-described antibody printing on the surface coupleable to the chamber, the slide 202 of the chip device 203 may include, printed on a same surface as the histological sample 201 is mounted, antibodies for binding to biological material of the histological sample 201.

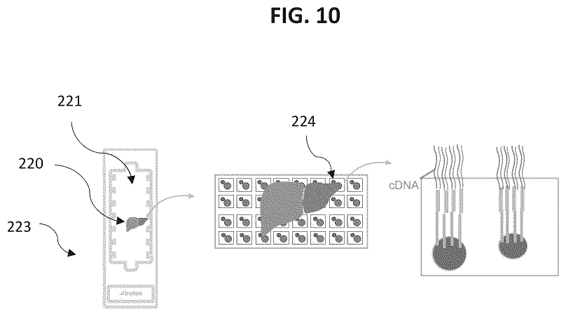

FIG. 10 is a schematic detailing a device and method for determining spatially resolved information from a sectioned histological sample. Histological sample 220 is mounted to a slide 221 of a chip device 223 comprising a plurality of chambers (see e.g., chamber 224), each comprising two capture beads configured to capture biological material such as nucleic acid sequences or proteins. Each chamber and the two capture beads residing in the chamber contact a discrete section of the sample and capture the biological material within that section of the sample, which provides spatial resolution as to the location in the histological sample from which the biological materials were captured. Captured biological material can then be detected, either via sequencing captured nucleic acids or by imaging detected proteins via labeled antibodies or detection agents. Capture beads configured for the capture of nucleic acids each have a unique predetermined chamber barcode sequence that is incorporated into synthesized cDNA sequences making it possible to track each sequence back to the chamber in which it was captured. Prior to the use of the device for the analysis of a histological sample, the chamber barcode sequence of the capture bead oligonucleotides is determined by “on chip,” or in chamber, sequencing.

Devices and methods of the disclosure permit high spatial resolution detection of biological material across the biological sample. Spatial resolution is determined according to the size of the chambers, the spacing between each individual chambers, and the size and number of capture beads present in each chamber. By way on non-limiting example, a 20 μm square chamber will capture a larger portion of a biological sample than a 5 μm square chamber. Thus, depending on the application, the size and spacing of the chambers can be tuned to fit a desired analytical output. In some embodiments, the chambers can be sized such that single cell resolution of the biological sample is achieved wherein each chamber and each bead within said chamber captures only biological material from a single cell. In some embodiments, the spatial resolution is between 0.1 μm and 200 μm, inclusive of all values and ranges in between. In certain embodiments, the spatial resolution is from 1 μm to 100 μm, 1 μm to 50 μm, or 5 μm to 50 μm, or 10 μm to 100 μm.

In some embodiments, the biological sample is a frozen or formaldehyde-fixed sectioned histological sample. In some embodiments, the biological sample is Formalin-Fixed Paraffin-Embedded (FFPE). In some embodiments, the sectioned histological sample is a tumor, an organ, healthy tissue, cancerous tissue, blood, or an embryo.

In embodiments, the sample can be an organ. The organ can be adrenal glands, heart, pancreas, thymus gland, testes, anus, hypothalamus, pineal gland, thyroid, epididymis, appendix, joints, parathyroid glands, trachea, vas deferens, bladder, kidneys, pharynx, tongue, seminal vesicles, bones, large intestine, pituitary gland, ureters, bulbourethral glands, bone marrow, larynx, prostate, urethra, penis, brain, liver, rectum, uterus, scrotum, bronchi, lungs, salivary glands, nerves, subcutaneous tissue, diaphragm, lymph nodes, skeletal muscles, ligaments, foramen ovale, ears, mammary glands, skin, tendons, arteries, oesophagus, mesentery, small intestine, clitoris, veins, eyes, mouth, spinal cord, vagina, capillaries, fallopian tubes, nasal cavity, spleen, vulva, lymphatic vessel, gallbladder, nose, stomach, cerebellum, tonsils, olfactory epithelium, ovary, teeth, placenta, or interstitium. In some embodiments, the biological sample is a tumor of an organ described herein.

The tumor can be a benign, pre-malignant or malignant tumor. In some embodiments, the tumor or cancerous tissue can be related to a cancer selected from: adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/ myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and/or Wilm' s Tumor.

In some embodiments, the biological sample is stained with an imaging agent. In some embodiments, the stain is H&E stain or an immunofluorescence stain. Such stains can be specific for an organelle, such as a cell nuclei, or a biomarker. In some embodiments, the stained sample can be imaged to detect the stained features in the sample. In some embodiments, the imaging occurs prior to treating the sectioned histological sample with a cell permeabilization reagent.

In some embodiments, the sample is treated with a permeabilization or lysis reagent. In some embodiments, the permeabilization or lysis reagent causes the sample to release biological material including, proteins, metabolites, and nucleic acids. In some embodiments, the sample is treated with the permeabilization or lysis reagent prior to mounting on the slide. In some embodiments, the sample is treated with the permeabilization or lysis reagent after being mounted on to the slide. In some embodiments, the sample is treated with the permeabilization or lysis reagent after coupling the slide to the surface comprising the plurality of chambers and capture beads. In some embodiments, the permeabilization or lysis reagent is a cell lysis buffer.

Methods of the disclosure provide devices configured to detect target biological molecules that can comprise at least one target protein or at least one target nucleic acid sequence. In some embodiments, the target biological molecules comprise at least one target protein and at least one target nucleic acid sequence. For methods of detecting nucleic acid sequences and proteins, each chamber of the plurality of chambers can comprise two capture beads, one configured to capture nucleic acid sequences and one configured to capture protein sequences. For methods of detecting nucleic acid sequences, devices can be configured to capture two distinct classes of nucleic acid, such as any combination of DNA and RNA. For example, devices of the disclosure can be configured such that a first capture bead (CB) is configured to capture mRNA and a second capture bead (CB) is configured to capture genomic DNA.

In some embodiments, the at least one target nucleic acid sequence is an RNA sequence. In some embodiments, the RNA sequence is an mRNA sequence.

In some embodiments, the first CB is configured to capture nucleic acid sequences, peptides, proteins, metabolites, or organic molecules. In some embodiments, the first CB comprises a capture moiety configured to capture nucleic acid sequences. In some embodiments, the first CB comprises a capture moiety configured to capture proteins.

In some embodiments, the device can further comprise a second capture bead. In some embodiments, the second CB is configured to capture nucleic acid sequences, peptides, proteins, metabolites, or organic molecules. In some embodiments, the second CB comprises a capture moiety configured to capture nucleic acid sequences. In some embodiments, the second CB comprises a capture moiety configured to capture proteins.

In some embodiments, each chamber of the plurality of chambers comprises a first CB and a second CB.

In some embodiments, the target nucleic acid sequences comprise from about one to about 1,000,000 target nucleic acid sequences. In some embodiments, the target proteins comprise from one to about 1,000,000 target proteins.

### Imaging and Quantifying Proteins

Proteins captured using capture beads of the disclosure configured to capture proteins are detected via visualization with a secondary antibody. The disclosure provides methods of detecting and quantifying proteins identified by forming complexes with protein capture moieties on capture beads of the disclosure. In some embodiments, following allowing the at least one capture antibody of the capture bead specific for the at least one protein to bind the at least one cellular protein forming at least one antibody:protein complex, substrate comprising at least one capture bead:protein complex is imaged. In some embodiments, imaging comprises detecting the fluorescent signal emitted by a secondary detection antibody. In some embodiments, the labeled secondary antibody comprises a fluorescent, gold or silver label. In some embodiments, the visualizing comprises contacting a first capture antibody:protein complex with a first labeled secondary antibody that binds the first capture antibody, contacting a second capture antibody:protein complex with a second labeled secondary antibody that binds the second capture antibody, and detecting the first labeled secondary antibody and the second labeled secondary antibody, wherein the first labeled secondary antibody and the second labeled secondary antibody each comprise a distinct label.

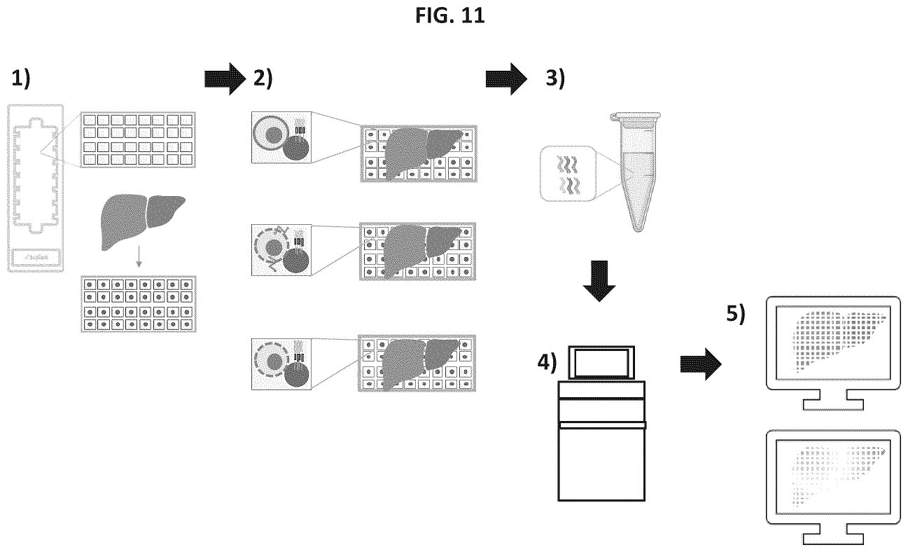

FIG. 11 is a schematic depicting an exemplary workflow wherein: 1) a slide mounted sectioned histological sample that is fixed and stained with H&E (hematoxylin and eosin) or an immunofluorescent stain is attached to a device comprising an array of chambers and capture beads, wherein each bead contains a unique barcode sequence that can be linked to the specific chamber in which the bead is located; 2) the sample is permeabilized such that biological material, such as nucleic acid sequences, is released into the chamber and is captured by the capture beads; 3) captured nucleic sequences are synthesized on device into cDNA sequences and collected before the collected sequences are prepared for downstream sequencing; 4) the collected nucleic acids, each comprising a unique barcode sequence, are sequenced; 5) the sequenced nucleic acids are analyzed and grouped by barcode sequence thereby enabling spatial analysis of genomic and/or transcriptomic analysis of the sectioned histologic sample.

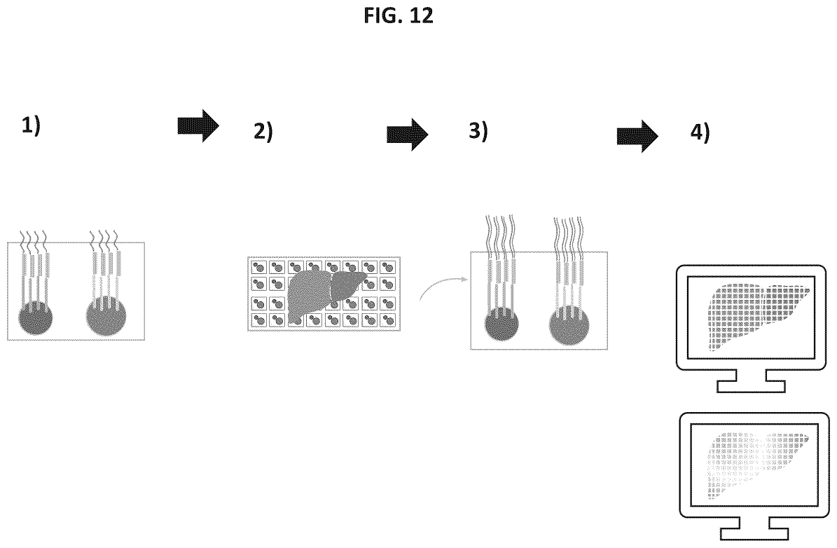

FIG. 12 is a schematic depicting: 1) the determination of capture bead barcode sequences derived prior to mating the histological sample to the array of chambers and capture beads enabling the location of each capture bead to be known prior to performing the assay. In other words, for each chamber of the plurality of chambers, the barcode sequence of each capture bead within each chamber is known. 2) The slide mounted sample is mated to the device comprising the plurality of chambers and capture beads. 3) The sample is permeabilized such that the capture beads can capture analytes released from the sample. 4) The captured analytes, in this case nucleic acid sequences, are sequenced and because each sequence comprises the barcode sequence it can be traced back to the well in which it was captured thereby enabling a spatial transcriptomic profile of the sample.

In some embodiments, the method further comprises quantifying the at least one protein. In some embodiments, the quantifying step comprises measuring an intensity and/or a density of the labeled secondary antibody. In some embodiments, detecting comprises determining the signal intensity to a signal associated with each antibody:protein complex. In some embodiments, the signal intensity associated with each antibody:protein complex is used to determine the level or concentration of each protein associated with each antibody:protein complex. Methods of detecting and quantifying the signal intensity of a detected antibody:protein complex, such as an antibody:protein complex is described in U.S. Pat. No. 10,584,366, the contents of which are incorporated by reference in their entirety.

### Sequencing Captured Nucleic Acid Sequences

The disclosure provides methods of sequencing target nucleic acid sequences captured by capture beads of the disclosure.

The disclosure provides methods of sequencing the individually unique chamber barcode sequence of capture beads configured to capture nucleic acids. In some embodiments, the individually unique chamber barcode sequence is sequenced on the substrate (e.g. “on chip) after the capture bead has been placed in the chamber of the disclosure. Sequencing on chip enables sequence information to be stored and correlated with the discrete chamber of the plurality of chambers, thereby linking the chamber with the individual bead carrying the individually unique chamber barcode sequence. Sequencing the individually unique chamber barcode sequence is comprises on chip fluorescent microscopy using fluorescently labeled nucleotide analogs.

In some embodiments, sequencing the individually unique chamber barcode sequence comprises synthesizing a cDNA barcode sequence. In some embodiments, the sequence encoding the individually unique chamber barcode sequence comprises 12 nucleotides. In some embodiments, the sequencing is performed in the chamber. In some embodiments, synthesizing the cDNA barcode sequence comprises contacting the sequence encoding the barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence. In some embodiments, the conditions sufficient for hybridization and cDNA synthesis comprise a plurality of deoxynucleotides (dNTPs), wherein the least one dNTP of the plurality of deoxynucleotides (dNTPs) comprises a modification. In some embodiments, each dNTP of the plurality of deoxynucleotides (dNTPs) comprises a modification. In some embodiments, the modification comprises a label, wherein the label comprises a fluorophore or a chromophore. In preferred embodiments, the label is a fluorescent label.

In some embodiments, each adenine comprises a first label, wherein each cytosine comprises a second label, each guanine comprises a third label, and each thymine comprises a fourth label. In some embodiments, the first label, the second label, the third label, and the fourth label are distinct labels that are spectrally-distinguishable fluorescent labels.

In some embodiments, sequencing the individually unique chamber barcode produces a cDNA barcode sequence that is linked to the discrete chamber in which the capture bead having the individually unique chamber barcode sequence originated.

The disclosure further provides methods of sequencing captured target nucleic acids. In some embodiments, sequencing the captured target nucleic acid sequences comprises synthesizing a cDNA sequence that incorporates the individually unique chamber barcode sequence and UMI into the cDNA sequence. In some embodiments, cDNA is synthesized from captured target nucleic acid sequences on chip and a template switch reaction is used to incorporate the individually unique chamber barcode, UMI, and captured target nucleic acid sequence into one stand.

In some embodiments, the cDNA sequences are analyzed. In some embodiments, the nucleic acid sequences are quantified via bioanalyzer. In some embodiments, analyzing comprises clustering the cDNA sequences by their barcode sequence. Clustering by barcode sequence enables all nucleic acids captured by a capture bead in an individual chamber to be traced back to that chamber and thus that section of the biological sample.

Methods for sequencing captured target nucleic acid sequences is described in U.S. patent application Ser. No. 16/349,183, published as US 2019/028526, the contents of which are incorporated by reference in their entirety.

CDNA sequences comprising the captured target nucleic acid sequences are amplified via PCR and used to generate libraries of nucleic acids suitable for downstream sequencing. In some embodiments, the sequencing comprises next generation sequencing.

### Definitions

Unless otherwise defined, scientific and technical terms used in connection with the disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The practice of embodiments of the inventions included in the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The following definitions are useful in understanding embodiments of disclosure:

The term “antibody” (Ab) as used herein includes monoclonal antibodies, polyclonal antibodies, multispecific antibodies (e.g., bispecific antibodies), and antibody fragments, as long as they exhibit the desired biological activity. The term “immunoglobulin” (Ig) is used interchangeably with “antibody” herein. In some embodiments, the antibody is an isolated antibody.

The term “about”, “approximately”, or “approximate”, when used in connection with a numerical value, means that a collection or range of values is included. In some embodiments, “about X” includes a range of values that are ±25%, ±20%, ±15%, ±10%, ±5%, ±2%, ±1%, ±0.5%, ±0.2%, or ±0.1% of X, where X is a numerical value. In some embodiments, the term “about” refers to a range of values which are 5% more or less than the specified value. In some embodiments, the term “about” refers to a range of values which are 2% more or less than the specified value. In some embodiments, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. In some embodiments, the expressions “x being an integer between 1 and 6” and “x being an integer of 1 to 6” both mean “x being 1, 2, 3, 4, 5, or 6”, i.e., the terms “between X and Y” and “range from X to Y, are inclusive of X and Y and the integers there between.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Capture antibodies of the disclosure may comprise one or more monoclonal antibodies. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

Monoclonal antibodies contemplated herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Capture antibodies of the disclosure may comprise humanized antibodies. A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed by substituting import hypervariable region sequences for the corresponding sequences of a human antibody. Accordingly, such “humanized” antibodies are chimeric antibodies wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

Capture antibodies of the disclosure may comprise intact antibodies. An “intact” antibody is one that comprises an antigen-binding site as well as a CL and at least heavy chain constant domains, CH 1, CH 2 and CH 3. The constant domains may be native sequence constant domains (e.g., human native sequence constant domains) or amino acid sequence variant thereof. Preferably, the intact antibody has one or more effector functions.

Capture antibodies of the disclosure may comprise an antibody fragment. An “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments.

Capture antibodies of the disclosure may comprise a functional fragment or an analog of an antibody. The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, FcεRI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH 1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Capture antibodies of the disclosure may comprise single-chain antibodies (also referred to as scFv). “Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

Capture antibodies of the disclosure may comprise diabodies. The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

Capture antibodies of the disclosure may comprise bispecific antibodies. In certain embodiments of the present disclosure, antibodies are bispecific or multi-specific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen.

Methods for making bispecific antibodies are known in the art. Traditional production of full length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10 6 M⁻¹, greater than or equal to about 10⁷ M⁻¹, or greater than or equal to 10⁸ M⁻¹. Affinity of an antibody for its cognate antigen is also commonly expressed as a dissociation constant KD, and in certain embodiments, an antibody specifically binds to a component of a secretome if it binds with a KD of less than or equal to 10⁻⁴ M, less than or equal to about 10⁻⁵ M, less than or equal to about 10⁻⁶ M, less than or equal to 10⁻⁷ M, or less than or equal 10⁻⁸ M. Affinities of antibodies can be readily determined using conventional techniques, for example, those described by Scatchard et al. (Ann. N.Y. Acad. Sci. USA 51:660 (1949)).

Subject and target cells of the disclosure may be isolated, derived, or prepared from any species, including any mammal. A “mammal” for purposes of treating n infection, refers to any mammal, including humans, domestic and farm animals, and zoo, sports, or pet animals, such as dogs, cats, cattle, horses, sheep, pigs, goats, rabbits, etc. Preferably, the mammal is human.

Subject cells of the disclosure may be used in a cellular therapy for the treatment of a disease or disorder. “Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal may be successfully “treated” when, after receiving a cellular therapy with a subject cell of the disclosure, the patient shows observable and/or measurable reduction in or absence of one or more of the following: reduction in one or more of the symptoms associated with disease or disorder; reduced morbidity and mortality, and improvement in quality of life issues. The above parameters for assessing successful treatment and improvement in the disease are readily measurable by routine procedures familiar to a physician. Methods of the disclosure may be used to determine the safety and/or efficacy of a cellular therapy before, during or after initiation of treatment of the subject.

Capture antibodies of the disclosure may be labeled to render them detectable using one or more means. “Label” as used herein refers to a detectable compound or composition that is conjugated directly or indirectly to the capture antibody so as to generate a “labeled” capture antibody. The label may be detectable by itself (e.g., a fluorescent label) or, in the case of an enzymatic label, may catalyze chemical alteration of a substrate compound or composition that is detectable.

Capture antibodies of the disclosure may selectively or specifically identify, capture, and/or quantify one or more small molecules in a secretome. A “small molecule” is defined herein to have a molecular weight below about 500 Daltons.

Capture antibodies of the disclosure may include nucleic acids or labeled nucleic acids. The terms “nucleic acid” and “polynucleotide” are used interchangeably herein to refer to single-or double-stranded RNA, DNA, or mixed polymers. Polynucleotides may include genomic sequences, extra-genomic and plasmid sequences, and smaller engineered gene segments that express, or may be adapted to express polypeptides.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof.

An “isolated polypeptide” is one that has been identified and separated and/or recovered from a component of its natural environment. In preferred embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature or can be produced by recombinant or synthetic means.

A polynucleotide “variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of some embodiments of the present disclosure, and evaluating one or more biological activities of the encoded polypeptide as described herein and/or using any of a number of techniques well known in the art.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of some embodiments of the present disclosure and evaluating one or more biological activities of the polypeptide as described herein and/or using any of a number of techniques well known in the art.

Modifications may be made in the structure of the polynucleotides and polypeptides of the disclosure and still obtain a functional molecule that encodes a variant or derivative polypeptide with desirable characteristics. When it is desired to alter the amino acid sequence of a polypeptide to create an equivalent, or even an improved, variant or portion of a polypeptide of some embodiments of the present disclosure, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences that encode said peptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. The substitution of like amino acids can be made effectively on the basis of hydrophilicity. The greatest local average hydrophilicity of a protein, as governed by the hydrophilicity of its adjacent amino acids, correlates with a biological property of the protein.

The following hydrophilicity values have been assigned to amino acid residues: arginine (+3.0); lysine (+3.0); aspartate (+3.0±1); glutamate (+3.0 ±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, CA; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, CA; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of some embodiments of the present disclosure. Software for performing BLAST analyses is publicly available through the National Center for Biotechnology Information.

“Homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least 70%, at least 75%, at least 80%, at least 90%, at least 95%, at least 98%, or at least 99% polynucleotide or polypeptide homology with a polynucleotide or polypeptide described herein.

### EXAMPLES

### Example 1: Capture Bead Substrate Preparation

The system of the disclosure (in this example, a polydimethylsiloxane (PDMS) substrate with an array of chambers containing capture beads) is prepared.

Bead loading. A 5 μL stock solution of capture beads is diluted in 35 μL of phosphate buffered saline (PBS). The bead solution is filtered through a 30 μm filter and washed with 5 mL of PBS to remove small bead fragments. The beads are then eluted from the filter and brought to a final volume of 35 μL. Prior to bead loading the substrate is plasma treated. The substrate is assembled into a flow cell with an acrylic slide and rubber gasket and secured against a bottom frame. The bead mixture is added to the flow cell and the bead suspension is mixed for an even distribution. The assembled substrate is rocked for 1.5 hrs and the flow cell is mixed every 30 mins. The mixture is then aspirated from the flow cell using an aspirator. PBS is then added to the flow cell and the assembled substrate is rocked for 30 mins. The mixture is then aspirated from the flow cell. Repeat the addition of PBS to the flow cell, rocking, and aspiration. The assembled substrate is incubated at 60 ° C. for 5 mins. The substrate is then disassembled from the flow cell and the substrate is kept horizontal. The substrate is incubated for 2 more mins at 60° C. Place the substrate on a spin coater and add 700 μL of 0.15% agarose across the substrate ensuring that it covers the entire surface to immobilize the beads. Spin coat the substrate using the following progression: 100 rpm for 10 sec, 300 rpm for 10 sec, 500 rpm for 10 sec, 700 rpm for 10 sec, 900 rpm for 10 sec, 1500 rpm for 10 sec, 2000 rpm for 10 sec, and 2500 rpm for 20 sec. The substrate is then dried for 1 hr. The chambers are then rehydrated with 5 mL of PBS. Excess PBS is removed by tilting the substrate. The substrate is then incubated for 5 mins at 60° C. followed by a 30 min rest at room temperature.

### Example 2: On-Device Barcode Reading of an Individually Unique Barcode Sequence of a Capture Bead

The unique barcode sequence of each capture bead of the array of chambers containing capture beads is read on device.

Pre-hybridize the beads with sequencing primer at 20 uM final concentration in PBS with 0.1% triton at a 40 μL volume and incubate for 5 min at room temperature, mixing once during incubation. Remove primer solution and flush twice with 100 μL Post Extension Buffer (PEB) (Table 3).

Prepare 50 uL of fluorescent sequencing master mix according to Table 1. Slowly dispense 45 μL of fluorescent reaction mix into the flow cell and incubate 5 minutes at room temperature. Transfer to an incubator for 20 min at 60° and mix gently every 5 mins. Wash once slowly with 90 uL PEB and remove buffer.

TABLE 1
Fluorescent sequencing master mix

| | 1 | 2 | 3 | 4 |
|---|---|---|---|---|
| Reagent | chip | chips | chips | chips |
| Thermopol buffer | 5.0 | 10.0 | 15.0 | 20.0 |
| (10×) | | | | |
| 200 mM KCl | 2.5 | 5.0 | 7.5 | 10.0 |
| Post Extension Buffer | 10.0 | 20.0 | 30.0 | 40.0 |
| 0.1M NaOH | 6.0 | 12.0 | 18.0 | 24.0 |
| 488A | 4 | 8 | 12 | 16 |
| 647T (1:10 dilute)* | 0.5 | 1.0 | 1.5 | 2.0 |
| 488C | 0.3 | 0.6 | 0.9 | 1.2 |
| 647C | 0.25 | 0.50 | 0.75 | 1.00 |
| fill G | 0.5 | 1.0 | 1.5 | 2.0 |
| 25 mM MgCl2 | 14.0 | 28.0 | 42.0 | 56.0 |
| Therminator X | 0.5 | 1.0 | 1.5 | 2.0 |
| polymerase | | | | |
| water | 4.5 | 8.9 | 13.4 | 17.8 |

*Use 1:15 dilution of 647T for 1^{st} cycle

Prepare 200 μL “Fill-in” reversible terminator master mix according to Table 2. Slowly dispense 45 μL of “fill in” reaction mix into the flow cell of the device containing the substrate with chambers containing capture beads and incubate for 5 min at room temperature. Transfer to an incubator for 20 min at 60° C. and mix gently every 5 min. Remove remaining reaction mixture and flush 2× with 90 μL PWB (Table 4), flush 1× with 90 μL PEB, and flush 1× with 90 μL PEB with BSA.

TABLE 2
Fill-in” reversible terminator master mix

| | 1 | 2 | 3 | 4 |
|---|---|---|---|---|
| Reagent | chips | chips | chips | chips |
| Thermopol buffer | 5.0 | 10 | 15 | 20 |
| (10×) | | | | |
| 200 mM KCl | 2.5 | 5 | 7.5 | 10 |
| Post Extension Buffer | 10.0 | 20 | 30 | 40 |
| 25 mM MgCl2 | 10.5 | 21 | 31.5 | 42 |
| 0.1M NaOH | 3.5 | 7 | 10.5 | 14 |
| fill ins* | 16.0 | 32 | 48 | 64 |
| Standard Therminator | 1.0 | 2 | 3 | 4 |
| polymerase | | | | |
| water | 1.5 | 3 | 4.5 | 6 |

*Volume is for total fill ins, divide the volume by 4 to get the volume per nucleotide

Image the device containing the substrate with chambers containing capture beads using bright field, 405 nm, 488 nm, 555, nm and 647 nm filters. Exposure time for 405 nm, 488 nm, 555 nm, and 647 nm may need to be lowered depending on bead signal. In some embodiments, the wavelength of each filter is ±3 nm.

Save the image for analysis. Each bead is given a location (chamber 1, chamber 2, chamber 3, etc.) Software determines nucleotide call based on fluorescent signal intensity of bead signal and records nucleotide call and bead location. Over the course of several cycles, the barcode sequence of each bead is determined and linked to their chamber location. Cleave 3′ reversible terminator with Buffered Sodium Nitrite. Buffered Nitrite solution formula: 700 mM NaNO2 from powder using 1M NaOAc ph5.5 and 0.1% Triton X. Check pH and adjust as necessary. 1mL NaOAc, 1 uL Triton X, 50 mg Sodium nitrite. Gently dispense 90 μL buffered Nitrite solution into the flow cell and incubate at room temp for 5 min, mixing halfway through incubation, then remove solution. Repeat the addition of 90μL buffered nitrite solution and incubation two additional times. Wash once with 90uL PEB and remove buffer.

Cleave fluorescent molecules with 1% sodium periodate. Gently dispense 90 μL sodium periodate solution into the flow cell and incubate at room temperature for 3 min, mixing halfway through incubation, followed by removal of the solution. Wash twice with 90 μL PWB, and twice with PEB, removing solution after each wash.

Repeat the protocol for each nucleotide of the barcode sequence, beginning at the addition of the fluorescent sequencing master mix to the device and ending with cleaving the fluorescent molecule with sodium periodate. Thus, for a 10 nucleotide barcode sequence, 10 cycles will be performed.

After barcode reading is complete and sequencing data is obtained, software must link the gene expression data for each barcode so that the mRNA expressed in an individual cell is connected to the physical location on the chip.

TABLE 3
PEB components

| Reagent | Volume for 25 μL | |
|---|---|---|
| 1M tris pH 8 | 1250 | μL |
| 30% MeONH2 | 83.3 | μL |
| 1M NaOH | 25 | μL |
| 100% Triton X | 25 | μL |
| water | 23.6 | mL |

TABLE 4
PWB components

| | volume for 25 mL | |
|---|---|---|
| 1M tris pH 7.5 | 1250 | μL |
| 0.5M EDTA | 25 | μL |
| 30% MeONH2 | 83.3 | μL |
| 5M NaCl | 5 | mL |
| 100% Triton X | 25 | μL |
| water | 18.6 | mL |

### Example 3: Generating cDNA Library from Captured mRNA

Captured mRNA sequences are synthesized into cDNA sequences containing the captured mRNA, chamber barcode sequence, and unique molecular identifier.

The assembled device containing the substrate with the array of chambers and capture beads is removed from the IsoLight device. Any remaining liquid is removed from the device via aspiration. The flow cell of the device is washed twice with 500 μL of PBS and removed followed by a single wash with 200 μL of RT buffer, which is removed following washing. Reverse transcription mixture according to Table 5 is applied to the flow cell in a 40 μL or 200 μL addition and mixed via pipette. The device is placed on to a thermal cycler and incubated for 10 min at 25 ° C. followed by incubation for 90 min at 45° C. The device is washed three times with 500 μL of PBS.

TABLE 5
Reverse Transcription Mixture

| | Volume |
|---|---|
| Reagent | (μL) |
| 5× Maxima RT buffer | 7.7 |
| 20% Ficol | 7.7 |
| 10 mM dNTPs | 3.8 |
| Rnase inhibitor | 1.0 |
| 100 uM Template switch oligo | 1.9 |
| Reverse Transcriptase | 1.9 |
| Water | 4.8 |
| extra 5× RT buffer | 9.6 |

### Example 4: Removing cDNA from Immobilized Capture Beads

Synthesized cDNA is removed from the chambers containing capture beads without disturbing or removing the capture beads.

Flush the device using two 300 μL additions of PBS aspirating PBS from the device after each addition. Dispense 40 μL of 60% DMSO in water into the flow cell and mix via pipetting. Incubate for 2 mins. Pipette to mix and collect sample into 1.5 mL tube. Add 20 μL of PBS to each tube. The DNA is cleaned and purified utilizing Solid Phase Reversible Immobilization (SPRI) beads from Beckman Coulter. Clean up is done according to Beckman Coulter Ampure XP manufacturer's protocol. Following clean up the resultant cDNA mixture comprises the cDNA generated in each chamber of the plurality of chambers.

### Example 5: Amplification of cDNA Library

The cDNA mixture is amplified utilizing two PCR steps to form a cDNA library. The eluted cDNA (10 μL volume) following SPRI clean up is amplified utilizing a PCR mixture according to Table 6. A 40 μL volume of PCR mixture is added to the cDNA sample and mixed. The thermal cycler is run according to the protocol in Table 7. Perform a 0.6X SPRI clean up to capture cDNA and concentrate into a smaller volume per manufacture protocol.

TABLE 6
PCR mix

| Reagent | μL |
|---|---|
| Jump Start mix (2×) | 25 |
| 100 uM SMART PCR primer | 0.8 |
| 100 uM primer 23′ | 0.8 |
| SPRI elution (cDNA) | 10 |
| water | 13.4 |

TABLE 7
Step 1 PCR Cycle

| Step | Temperature | Time | |
|---|---|---|---|
| 1 | 95° C. | 3 | min |
| 2 | 98° C. | 20 | sec |
| 3 | 65° C. | 45 | sec |
| 4 | 72° C. | 3 | min |
| 5 | go to step 2 for 4 | | |
| | cycles | | |
| 6 | 98° C. | 20 | sec |
| 7 | 67° C. | 20 | sec |
| 8 | 72° C. | 3 | min |
| 9 | go to step 6 for 8 | | |
| | cycles | | |
| 10 | 72° C. | 5 | min |

Hold at 10° C.

A second round of PCR is conducted to generate the cDNA library. The eluted cDNA (10 μL volume) following SPRI clean up is amplified utilizing a PCR mixture according to Table 6. A 40 μL volume of PCR mixture is added to the cDNA sample and mixed. The thermal cycler is run according to the protocol in Table 8. Perform a 1X SPRI clean up to capture cDNA and concentrate into a smaller volume per manufacture protocol.

TABLE 8
Step 2 PCR Cycle

| Step | Temperature | Time | |
|---|---|---|---|
| 1 | 94° C. | 2 | min |
| 2 | 94° C. | 30 | sec |
| 3 | 55° C. | 30 | sec |
| 4 | 72° C. | 2 | min |
| 5 | go to step 2 | | |
| | repeat 21× | | |
| 6 | 72° C. | 5 | min |

hold at 10° C.

The amplified cDNA is run on an Agilent High Sensitivity DNA BioAnalyzer chip (in this example) using a High Sensitivity DNA kit, per manufacturer protocol. The library is now ready for sequencing.

Notwithstanding the appended claims, the disclosure sets forth the following embodiments.

The disclosure provides a multiplex assay chip device configured for multiplexed analysis of biological material, the device comprising: a plurality of capture beads (CB) each bead including a capture moiety; a substrate having a plurality of chambers each including: an open end arranged on a first side of the substrate, and a length, a width, and a depth (“dimensions”), at least one CB arranged within each chamber; and a surface configured to couple with the first side of the substrate to cover each chamber; wherein the surface is capable of being mounted with a biological sample.

The disclosure provides a multiplex assay chip device configured for multiplexed analysis of biological material, the device comprising: a plurality of capture beads (CB) each bead including a capture moiety; a substrate having a plurality of chambers each including: an open end arranged on a first side of the substrate, and a length, a width, and a depth (“dimensions”), at least two CBs arranged within each chamber; wherein each CB includes a CB diameter smaller than the pocket diameter; and a surface configured to couple with the first side of the substrate to cover each chamber; wherein the surface is capable of being mounted with a biological sample.

The disclosure provides a multiplex assay chip device configured for multiplexed analysis of biological material, the device comprising: a plurality of capture beads (CB) each bead including a capture moiety; a substrate having a plurality of chambers each including: an open end arranged on a first side of the substrate, and a length, a width, and a depth (“dimensions”), a pocket P1 having a pocket diameter; and at least one CB arranged within the pocket of each chamber; wherein each CB includes a CB diameter smaller than the pocket diameter; and a surface configured to couple with the first side of the substrate to cover each chamber wherein the surface is capable of being mounted with a biological sample.

The disclosure provides a multiplex assay chip device configured for multiplexed analysis of biological material, the device comprising: a plurality of capture beads (CB) each bead including a capture moiety; a substrate having a plurality of chambers each including: an open end arranged on a first side of the substrate, and a length, a width, and a depth (“dimensions”), a plurality of pockets comprising at least a first pocket P1 and a second pocket P2; and at least one CB arranged within each pocket of each chamber; wherein each CB includes a CB diameter smaller than the pocket diameter; and a surface configured to couple with the first side of the substrate to cover each chamber wherein the surface is capable of being mounted with a biological sample.

In some embodiments, P1 and P2 have the same pocket diameter. In some embodiments, P1 and P2 have different pocket diameters. In some embodiments, each chamber has a width of between 1 and 100 μm. In some embodiments, the chamber has a length of between 1 and 2000 μm. In some embodiments, the chamber has a depth of between 1 and 100 μm. In some embodiments, the distance between adjacent chambers is between 0.01 and 10 μm.

In some embodiments, P1 has a pocket diameter of between 5 and 50 μm larger than the chamber width. In some embodiments, P1 has a pocket diameter of between 10 and 100 μm. In some embodiments, P2 has a pocket diameter of between 5 and 50 μm larger than the P1 pocket diameter. In some embodiments, P2 has a pocket diameter of between 15 and 150 μm.

In some embodiments, the diameter of the CB of P1, CB1, is between 0 and 50 μm smaller than the diameter of P1. In some embodiments, the diameter of the CB of P2, CB2, is between 0 and 50 μm smaller than the diameter of P2. In some embodiments, the diameter of the CB in P2, CB2 is larger than the diameter of P1. In some embodiments, the CB of P2, CB2, does not fit within Pl.

In some embodiments, P1 and P2 are center aligned with respect the width of the chamber. In some embodiments, P1 and P2 are not center aligned with respect the width of the chamber. In some embodiments, P1 and P2 are non-overlapping. In some embodiments, P1 and P2 are positioned at any point along the length of the chamber.

In some embodiments, P1 and P2 are any shape. In some embodiments, P1 and P2 are circular, ovoid, rectangular, square, triangular, pentagonal, hexagonal, or octagonal.

In some embodiments, the plurality of pockets includes a third pocket P3 and a third CB, CB3. In some embodiments, P3 comprises a pocket diameter of between 5 and 50 μm larger than the P2 pocket diameter. In some embodiments, P3 comprises a pocket diameter of between 20 and 200 μm. In some embodiments, CB3 comprises a diameter of between 0 and 50 μm smaller than the P3 pocket diameter.

In some embodiments, the CB capture moiety is configured to capture nucleic acid sequences, peptides, proteins, metabolites, or organic molecules. In some embodiments, the CB capture moiety is configured to capture nucleic acid sequences. In some embodiments, the captured nucleic acid sequence is DNA, RNA, or a combination thereof. In some embodiments, the DNA is autosomal DNA, chromosomal DNA, cDNA, exosome DNA, single stranded DNA, or double stranded DNA.

In some embodiments, the RNA is mRNA, rRNA, tRNA, snRNA, regulatory RNA, microRNA, exosome RNA, or double stranded RNA. In some embodiments, the RNA is an mRNA.

In some embodiments, the RNA is a guide RNA from a CRISPR-Cas system.

In some embodiments, the nucleic acid capturing CB is an oligonucleotide capture bead comprising a nucleic acid capture sequence tethered to a bead. In some embodiments, the nucleic acid capturing CB comprises from one to 10,000,000 capture nucleic acid sequences.

In some embodiments, the nucleic acid capturing CB capture nucleic acid sequence comprises an individually unique chamber barcode sequence, a PCR handle, a unique molecular identifier (UMI), a barcode handle sequence, and a capture sequence. In some embodiments, the capture sequence comprises a polyT sequence for mRNA polyA capture.

In some embodiments, the capture sequence comprises an rGrGrG capture sequence for mRNA capture. In some embodiments, the capture sequence comprises a gene-specific or sequence-specific capture sequence.

In some embodiments, each nucleic acid sequence of the nucleic acid capturing CB comprises a unique UMI. In some embodiments, the barcode sequence of the CB is unique to each CB of the plurality of CBs. In some embodiments, the barcode sequence of the CB is unique to each chamber of the plurality of chambers.

In some embodiments, the CB capture moiety is configured to capture proteins.

In some embodiments, the protein capturing CB comprises a capture antibody tethered to a bead.

In some embodiments, the bead comprises plastic, polymer, metal, or silica. In some embodiments, the bead is porous. In some embodiments, the porous bead is configured to release one or more agents. In some embodiments, the one or more agents can comprise an enzyme, catalyst, stimulatory agent, or therapeutic agent.

In some embodiments, the surface comprises glass.

In some embodiments, the chambers are circular, ovoid, square, rectangular, pentagonal, hexagonal, heptagonal, octagonal, or nonagonal in shape. In some embodiments, the chambers are irregular in shape. In some embodiments, the plurality of chambers comprises between 2 and 200,000 chambers.

In some embodiments, the plurality of chambers comprises between 12,000 and 200,000 chambers. In some embodiments, the plurality of chambers comprises between 12,000 and 100,000 chambers. In some embodiments, the plurality of chambers comprises between 12,000 and 75,000 chambers. In some embodiments, the plurality of chambers comprises between 20,000 and 200,00 chambers. In some embodiments, the plurality of chambers comprises between 20,000 and 100,000 chambers.

In some embodiments, the substrate comprises a polymer. In some embodiments, the polymer comprises polydimethylsiloxane (PDMS).

In some embodiments, the biological material is a biological sample, a metabolite, a protein, a polypeptide, or a cell. In some embodiments, the biological sample is a blood sample. In some embodiments, the biological sample is healthy tissue, cancerous tissue, a tumor, an organ, blood, or an embryo. In some embodiments, the biological sample is a sectioned histological sample. In some embodiments, the sectioned histological sample is frozen or formaldehyde fixed.

The disclosure provides a method for determining spatially resolved information from a biological sample, comprising: I) providing a device comprising a substrate having a plurality of chambers each comprising at least one first capture bead (CB); II) obtaining a sectioned histological sample from a subject mounted to a surface; III) treating the sectioned histological sample with a cell lysis or permeabilization reagent, under conditions sufficient for target biological molecules to be released from the sample; IV) coupling the surface to the substrate such that the surface seals the plurality of chambers forming a plurality of enclosures; wherein each enclosure comprises a first CB in fluid communication with a portion of the sectioned histological sample and target biological molecules; V) incubating the sample under conditions sufficient for the target biological molecules to contact the CB to form CB-target biological molecule complexes; and VI) detecting the complexed target biological molecule.

The disclosure provides a method for determining spatially resolved information from a biological sample, comprising: I) providing a device comprising a substrate having a plurality of chambers each comprising at least one first capture bead (CB); II) obtaining a sectioned histological sample from a subject mounted to a surface; III) treating the sectioned histological sample with a cell lysis or permeabilization reagent, under conditions sufficient for target nucleic acid sequences to be released from the sample; IV) coupling the surface to the substrate such that the surface seals the plurality of chambers forming a plurality of enclosures; wherein each enclosure comprises a first CB in fluid communication with a portion of the sectioned histological sample and target nucleic acid sequences; V) incubating the sample under conditions sufficient for the target nucleic acid sequences to contact the CB to form CB-target nucleic acid sequence complexes; and VI) sequencing the complexed target nucleic acid sequences.

In some embodiments, the biological sample is a frozen or formaldehyde-fixed sectioned histological sample. In some embodiments, the sectioned histological sample is stained with H&E stain or an immunofluorescence stain. In some embodiments, the immunofluorescence stain is specific for a biomarker or cellular organelle.

In some embodiments, the method further comprising imaging the stained sectioned histological sample. In some embodiments, the imaging occurs prior to treating the sectioned histological sample with the cell permeabilization reagent.

In some embodiments, the sectioned histological sample is healthy tissue, cancerous tissue, a tumor, an organ, blood, or an embryo.

In some embodiments, the cell permeabilization reagent is a cell lysis reagent.

In some embodiments, the target biological molecules comprise at least one target protein or at least one target nucleic acid sequence. In some embodiments, the target biological molecules comprise at least one target protein and at least one target nucleic acid sequence. In some embodiments, the at least one target nucleic acid sequence is an RNA sequence. In some embodiments, the RNA sequence is an mRNA sequence.

In some embodiments, the first CB is configured to capture nucleic acid sequences, peptides, proteins, metabolites, or organic molecules. In some embodiments, the first CB comprises a capture moiety configured to capture nucleic acid sequences. In some embodiments, the first CB comprises a capture moiety configured to capture proteins.

In some embodiments, the method further comprising at least one second capture bead. In some embodiments, the second CB is configured to capture nucleic acid sequences, peptides, proteins, metabolites, or organic molecules. In some embodiments, the second CB comprises a capture moiety configured to capture nucleic acid sequences. In some embodiments, the second CB comprises a capture moiety configured to capture proteins.

In some embodiments, each chamber of the plurality of chambers comprises a first CB and a second CB.

In some embodiments, the first CB and the second CB are configured to capture different biological molecules.

In some embodiments, the target nucleic acid sequences comprise from about one to about 1,000,000 target nucleic acid sequences. In some embodiments, the target protein comprise from one to about 1,000,000 target proteins.

In some embodiments, the CB comprises a plurality of nucleic acid sequences, each comprising an individually unique barcode sequence comprising a predetermined number of base pairs, a PCR handle, a unique molecular identifier (UMI), a barcode handle sequence, and a capture sequence.

In some embodiments, the CB comprises from one to 10,000,000 nucleic acid sequences. In some embodiments, the barcode sequence of the CB is unique to each CB of the plurality of CBs. In some embodiments, the barcode sequence of the CB is unique to each chamber of the plurality of chambers. In some embodiments, each nucleic acid sequence of the CB comprises a unique UMI. In some embodiments, the individually unique barcode sequence of the CB is sequenced.

In some embodiments, sequencing the individually unique barcode sequence comprises synthesizing a cDNA barcode sequence. In some embodiments, synthesizing the cDNA barcode sequence comprises contacting the sequence encoding the barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence. In some embodiments, an individually unique chamber barcode sequence of the CB is sequenced.

In some embodiments, sequencing the individually unique chamber barcode sequence comprises synthesizing a cDNA barcode sequence. In some embodiments, synthesizing the cDNA barcode sequence comprises contacting the sequence encoding a barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence. In some embodiments, the sequence encoding the barcode comprises 12 nucleotides.

In some embodiments, the conditions sufficient for hybridization and cDNA synthesis comprise a plurality of deoxynucleotides (dNTPs). In some embodiments, at least one dNTP of the plurality of deoxynucleotides (dNTPs) comprises a modification. In some embodiments, each dNTP of the plurality of deoxynucleotides (dNTPs) comprises a modification.

In some embodiments, the modification comprises a label. In some embodiments, the label comprises a fluorophore or a chromophore. In some embodiments, the label is a fluorescent label.

In some embodiments, each adenine comprises a first label, wherein each cytosine comprises a second label, each guanine comprises a third label, and each thymine comprises a fourth label. In some embodiments, the first label, the second label, the third label, and the fourth label are distinct labels. In some embodiments, the first label, the second label, the third label, and the fourth label are spectrally distinguishable fluorescent labels.

In some embodiments, the nucleic acid encoding the barcode further comprises a sequence encoding a TSO hybridization site. In some embodiments, the TSO hybridization site comprises a poly-rG sequence. In some embodiments, the method further comprising contacting the nucleic acid sequence encoding the barcode of the CB and a TSO under conditions sufficient for hybridization of the TSO to a portion of the nucleic acid encoding the barcode to produce a nucleic acid/TSO duplex.

In some embodiments, the TSO comprises a sequence complementary to the sequence encoding the UMI, a sequence complementary to the sequence encoding the TSO handle, a sequence complementary to the sequence encoding the sequence encoding a TSO hybridization site, and a sequence complementary to the target nucleic acid sequences.

In some embodiments, sequencing comprises synthesizing a cDNA sequence comprising one of the complexed target nucleic acid sequences for each of the complexed target nucleic acid sequences. In some embodiments, the cDNA sequence comprises the target nucleic acid sequence, UMI, and individually unique chamber barcode sequence.

In some embodiments, sequencing comprises removing the cDNA sequences from the chamber. In some embodiments, sequencing comprises amplifying the cDNA sequences by PCR.

In some embodiments, the sequencing method is next generation sequencing (NGS). In some embodiments, the method further comprising analyzing the cDNA sequences. In some embodiments, the method further comprising analyzing the cDNA sequences. In some embodiments, the cDNA sequences are clustered by barcode sequence. In some embodiments, the cDNA sequences are quantified by bioanalyzer.

In some embodiments, the target protein contacts the bead to form a CB-target protein complex.

In some embodiments, the detecting the target protein comprises contacting the CB-target protein complex with a labeled secondary antibody and imaging the labeled secondary antibody.

In some embodiments, the spatial resolution is 0.1 μm to 100 μm.

In some embodiments, the spatial resolution is single cell.

In some embodiments, an individually unique chamber barcode sequence of the CB is sequenced. In some embodiments, sequencing the individually unique chamber barcode sequence comprises synthesizing a cDNA barcode sequence. In some embodiments, synthesizing the cDNA barcode sequence comprises contacting the sequence encoding a barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence. In some embodiments, the sequence encoding the barcode comprises 12 nucleotides. In some embodiments, the conditions sufficient for hybridization and cDNA synthesis comprise a plurality of deoxynucleotides (dNTPs). In some embodiments, at least one dNTP of the plurality of deoxynucleotides (dNTPs) comprises a modification. In some embodiments, each dNTP of the plurality of deoxynucleotides (dNTPs) comprises a modification.

In some embodiments, the modification comprises a label. In some embodiments, the label comprises a fluorophore or a chromophore. In some embodiments, the label is a fluorescent label. In some embodiments, each adenine comprises a first label, wherein each cytosine comprises a second label, each guanine comprises a third label, and each thymine comprises a fourth label. In some embodiments, the first label, the second label, the third label, and the fourth label are distinct labels. In some embodiments, the first label, the second label, the third label, and the fourth label are spectrally distinguishable fluorescent labels.

In some embodiments, the nucleic acid encoding the barcode further comprises a sequence encoding a TSO hybridization site. In some embodiments, the sequence encoding a TSO hybridization site comprises a poly-riboguanine (poly-rG) sequence. In some embodiments, further comprising contacting the nucleic acid sequence encoding the barcode of the CB and a TSO under conditions sufficient for hybridization of the TSO to a portion of the nucleic acid encoding the barcode to produce a nucleic acid/TSO duplex. In some embodiments, the TSO comprises a sequence complementary to the sequence encoding the UMI, a sequence complementary to the sequence encoding the TSO handle, a sequence complementary to the sequence encoding the sequence encoding a TSO hybridization site, and a sequence complementary to the target nucleic acid sequences. In some embodiments, sequencing comprises synthesizing a cDNA sequence comprising one of the complexed target nucleic acid sequences for each of the complexed target nucleic acid sequences. In some embodiments, the cDNA sequence comprises the target nucleic acid sequence, UMI, and individually unique chamber barcode sequence.

In some embodiments, sequencing comprises removing the cDNA sequences from the chamber. In some embodiments, sequencing comprises amplifying the cDNA sequences by PCR. In some embodiments, the sequencing method is next generation sequencing (NGS). In some embodiments, further comprising analyzing the cDNA sequences.

As used in this specification and the appended claims, the singular forms “a,” “an” and “the” include plural references unless the content clearly dictates otherwise.

While various inventive embodiments have been described and illustrated herein, those of ordinary skill in the art will readily envision a variety of other means, structures, configurations, components, and the like, for performing a function, a step, and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, compounds, steps, and configurations described herein are meant to be merely an example and that the actual parameters, dimensions, materials, compounds steps, and configurations will depend upon the specific application or applications for which the inventive teachings is/are used. Those skilled in the art will recognize or be able to ascertain using no more than routine experimentation, many equivalents to the specific inventive embodiments described herein. It is therefore to be understood that the foregoing embodiments are presented by way of example only and that, within the scope of claims supported by the subject disclosure and equivalents thereto, and inventive embodiments may be practiced otherwise than as specifically described and claimed. Inventive embodiments of the present disclosure are directed to each individual feature, device, system, article, material, kit, step, function/functionality, and method described herein. In addition, any combination of two or more such features, devices, systems, articles, materials, kits, steps, functions/functionality, and methods, if such features, systems, articles, materials, kits, steps, functions/functionality, and methods are not mutually inconsistent, is included within the inventive scope of the present disclosure and considered embodiments.

Embodiments disclosed herein may also be combined with one or more features, as well as complete systems, devices, and/or methods, known in the art, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as noted, various inventive concepts may be embodied as one or more methods, of which one or more examples have been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A multiplex assay chip device configured for multiplexed analysis of biological material, the device comprising:
a plurality of capture beads (CB), each bead including a CB capture moiety and having a diameter;
a substrate having a plurality of chambers, each of the plurality of chambers including an open end arranged on a first side of the substrate,
at least one first region having a first length, a first width, and a first depth, wherein each of the first length, the first width, and the first depth is greater than the bead diameter,
at least one second region having a second length, a second width, and a second depth, wherein at least one of the second length, the second width, and the second depth is less than the bead diameter;
at least one CB arranged within the at least one first region of each of the plurality of chambers of the substrate; and
a surface removably couplable to the first side of the substrate, wherein each of the plurality of chambers is covered when the surface is removably coupled to the first side of the substrate.

2. The device of claim 1, further comprising at least one substrate capture moiety attached to a surface of the at least one second region.

3. The device of either one of claim 1 or 2, wherein the at least one first region is two first regions and each one of the two first regions have different dimensions.

4. The device of any one of claims 1-3, wherein the at least one second region has a width of between 1 μm and 100 μm.

5. The device of any one of claims 1-4, wherein the at least one second region has a length of between 1 μm and 2000 μm.

6. The device of any one of claims 1-5, wherein the at least one second region has a depth of between 1 μm and 100 μm.

7. The device of any one of claims 1-6, wherein the distance between adjacent ones of the plurality of chambers is between 0.01 μm and 10 μm.

8. The device of any one of claims 1-7, wherein the first width of the at least one first region is between 5 μm and 50 μm larger than the width of the second width of the at least one second region.

9. The device of any one of claims 1-8, wherein the at least one first region is cylindrical and each of the first length and the first width are between 10 and 100 μm.

10. The device of any one of claims 3-9, wherein a second one of the two first regions has a dimension between 5 μm and 50 μm larger than a corresponding dimension of the first one of the first wo regions.

11. The device of any one of claims 3-10, wherein the second one of the two first regions has a dimension of between 15 1.μm and 150 μm.

12. The device of any one of claims 3-11, wherein a diameter of a CB capture moiety within the first one of the two first regions is between 0 μm and 50 μm smaller than a dimension of the first one of the two first regions.

13. The device of any one of claims 3-12, wherein a diameter of a CB capture moiety within the second one of the two first regions is between 0 μm and 50 μm smaller than a dimension of the second one of the two first regions.

14. The device of any one of claims 3-13, wherein the diameter of the CB capture moiety within the first one of the two first regions is larger than the diameter of the CB capture moiety within the second one of the two first regions.

15. The device of any one of claims 1-14, wherein a cross-section of the at least one first region is one or more of circular, ovoid, rectangular, square, triangular, pentagonal, hexagonal, and octagonal.

16. The device of any one of the preceding claims, wherein the CB capture moiety is an oligonucleotide capture bead comprising a nucleic acid capture sequence tethered to the CB.

17. The device of any one of the preceding claims, wherein the nucleic acid capture sequence of the CB capture moiety comprises an individually unique chamber barcode sequence, a PCR handle, a unique molecular identifier (UMI), a barcode handle sequence, and a capture sequence.

18. The device of any one of the preceding claims, wherein each nucleic acid capture sequence of the CB capture moiety comprises a unique UMI.

19. The device of any one of the preceding claims, wherein the barcode sequence of the CB capture moiety is unique to each chamber of the plurality of chambers.

20. The device of any one of the preceding claims, wherein the at least one substrate capture moiety is an antibody.

21. The device of claim 44, wherein the CB capture moiety comprises an antibody tethered to the CB.

22. The device of any one of the preceding claims, wherein the surface comprises glass.

23. The device of any one of the preceding claims, wherein the plurality of chambers comprises between 10,000 chambers and 100,000 chambers.

24. The device of any one of the preceding claims, wherein the substrate comprises a polymer.

25. The device of any one of the preceding claims, wherein the polymer comprises polydimethylsiloxane.

26. A method for determining spatially resolved information from a biological sample, comprising:
(a) obtaining a device according to claim 1;
(b) removing the surface from the substrate;
(c) mounting a histological sample to the surface;
(d) treating the histological sample with a cell lysis or permeabilization reagent, under conditions sufficient for target biological molecules to be released from the histological sample;
(e) coupling the surface to the substrate to cover the plurality of chambers, wherein the histological sample is disposed between the substrate and the surface, and wherein the surface seals the plurality of chambers forming a plurality of enclosures that are fluidicly isolated from each other;
(f) incubating the histological sample under conditions sufficient for the target biological molecules to form complexes with the CB capture moiety and the substrate capture moiety; and
(g) detecting the complexes.

27. A method for determining spatially resolved information from a biological sample, comprising:
(a) providing a device comprising a substrate having a plurality of chambers each comprising at least one first capture bead (CB);
(b) obtaining a sectioned histological sample from a subject mounted to a surface;
(c) treating the sectioned histological sample with a cell lysis or permeabilization reagent, under conditions sufficient for target biological molecules to be released from the sample;
(d) coupling the surface to the substrate such that the surface seals the plurality of chambers forming a plurality of enclosures; wherein each enclosure comprises a first CB in fluid communication with a portion of the sectioned histological sample and target biological molecules;
(e) incubating the sample under conditions sufficient for the target biological molecules to contact the CB to form CB-target biological molecule complexes; and
(f) detecting the complexed target biological molecule.

28. A method for determining spatially resolved information from a biological sample, comprising:
(a) providing a device comprising a substrate having a plurality of chambers each comprising at least one first capture bead (CB);
(b) obtaining a sectioned histological sample from a subject mounted to a surface;
(c) treating the sectioned histological sample with a cell lysis or permeabilization reagent, under conditions sufficient for target nucleic acid sequences to be released from the sample;
(d) coupling the surface to the substrate such that the surface seals the plurality of chambers forming a plurality of enclosures; wherein each enclosure comprises a first CB in fluid communication with a portion of the sectioned histological sample and target nucleic acid sequences;
(e) incubating the sample under conditions sufficient for the target nucleic acid sequences to contact the CB to form CB-target nucleic acid sequence complexes; and
(f) sequencing the complexed target nucleic acid sequences.

29. The method of any one of claim 27 or 28, wherein the biological sample is a frozen or formaldehyde-fixed sectioned histological sample.

30. The method of any one of claims 27-29, wherein the sectioned histological sample is stained with H&E stain or an immunofluorescence stain.

31. The method of any one of claims 27-30, wherein the immunofluorescence stain is specific for a biomarker or cellular organelle.

32. The method of any one of claims 27-31, further comprising imaging the stained sectioned histological sample.

33. The method of any one of claims 27-32, wherein the imaging occurs prior to treating the sectioned histological sample with the cell permeabilization reagent.

34. The method of any one of claims 27-33, wherein the sectioned histological sample is healthy tissue, cancerous tissue, a tumor, an organ, blood, or an embryo.

35. The method of any one of claims 27-34, wherein the cell permeabilization reagent is a cell lysis reagent.

36. The method of any one of claims 27-35, wherein the target biological molecules comprise at least one target protein or at least one target nucleic acid sequence.

37. The method of any one of claims 27-36, wherein the target biological molecules comprise at least one target protein and at least one target nucleic acid sequence.

38. The method of any one of claims 27-37, wherein the at least one target nucleic acid sequence is an RNA sequence.

39. The method of any one of claims 27-38, wherein the RNA sequence is an mRNA sequence.

40. The method of any one of claims 27-39, wherein the first CB is configured to capture nucleic acid sequences, peptides, proteins, metabolites, or organic molecules.

41. The method of any one of claims 27-40, wherein the first CB comprises a capture moiety configured to capture nucleic acid sequences.

42. The method of any one of claims 27-41, wherein the first CB comprises a capture moiety configured to capture proteins.

43. The method of any one of claims 27-42, further comprising at least one second capture bead.

44. The method of any one of claims 27-43, wherein the second CB is configured to capture nucleic acid sequences, peptides, proteins, metabolites, or organic molecules.

45. The method of any one of claims 27-44, wherein the second CB comprises a capture moiety configured to capture nucleic acid sequences.

46. The method of any one of claims 27-45, wherein the second CB comprises a capture moiety configured to capture proteins.

47. The method of any one of claims 27-46, wherein each chamber of the plurality of chambers comprises a first CB and a second CB.

48. The method of any one of claims 27-47, wherein the first CB and the second CB are configured to capture different biological molecules.

49. The method of any one of claims 27-48, wherein the target nucleic acid sequences comprise from about one to about 1,000,000 target nucleic acid sequences.

50. The method of any one of claims 27-49, wherein the target protein comprises from one to about 1,000,000 target proteins.

51. The method of any one of claims 27-50, wherein the CB comprises a plurality of nucleic acid sequences, each comprising an individually unique barcode sequence comprising a predetermined number of base pairs, a PCR handle, a unique molecular identifier (UMI), a barcode handle sequence, and a capture sequence.

52. The method of any one of claims 27-51, wherein the CB comprises from one to 10,000,000 nucleic acid sequences.

53. The method of any one of claims 27-52, wherein the barcode sequence of the CB is unique to each CB of the plurality of CBs.

54. The method of any one of claims 27-53, wherein the barcode sequence of the CB is unique to each chamber of the plurality of chambers.

55. The method of any one of claims 27-54, wherein each nucleic acid sequence of the CB comprises a unique UMI.

56. The method of any one of claims 27-55, wherein the individually unique barcode sequence of the CB is sequenced.

57. The method of any one of claims 27-56, wherein sequencing the individually unique barcode sequence comprises synthesizing a cDNA barcode sequence.

58. The method of any one of claims 27-57, wherein synthesizing the cDNA barcode sequence comprises contacting the sequence encoding the barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence.

59. The method of any one of claims 27-58, wherein an individually unique chamber barcode sequence of the CB is sequenced.

60. The method of any one of claims 27-59, wherein sequencing the individually unique chamber barcode sequence comprises synthesizing a cDNA barcode sequence.

61. The method of any one of claims 27-60, wherein synthesizing the cDNA barcode sequence comprises contacting the sequence encoding a barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence.

62. The method of any one of claims 27-61, wherein the sequence encoding the barcode comprises 12 nucleotides.

63. The method of any one of claims 27-62, wherein the conditions sufficient for hybridization and cDNA synthesis comprise a plurality of deoxynucleotides (dNTPs).

64. The method of any one of claims 27-63, wherein at least one dNTP of the plurality of deoxynucleotides (dNTPs) comprises a modification.

65. The method of any one of claims 27-64, wherein each dNTP of the plurality of deoxynucleotides (dNTPs) comprises a modification.

66. The method of any one of claims 27-65, wherein the modification comprises a label.

67. The method of any one of claims 27-66, wherein the label comprises a fluorophore or a chromophore.

68. The method of any one of claims 27-67, wherein the label is a fluorescent label.

69. The method of any one of claims 27-68, wherein each adenine comprises a first label, wherein each cytosine comprises a second label, each guanine comprises a third label, and each thymine comprises a fourth label.

70. The method of any one of claims 27-69, wherein the first label, the second label, the third label, and the fourth label are distinct labels.

71. The method of any one of claims 27-70, wherein the first label, the second label, the third label, and the fourth label are spectrally distinguishable fluorescent labels.

72. The method of any one of claims 27-71, wherein the nucleic acid encoding the barcode further comprises a sequence encoding a TSO hybridization site.

73. The method of any one of claims 27-72, wherein the sequence encoding a TSO hybridization site comprises a poly-riboguanine (poly-rG) sequence.

74. The method of any one of claims 27-73, further comprising contacting the nucleic acid sequence encoding the barcode of the CB and a TSO under conditions sufficient for hybridization of the TSO to a portion of the nucleic acid encoding the barcode to produce a nucleic acid/TSO duplex.

75. The method of any one of claims 27-74, wherein the TSO comprises a sequence complementary to the sequence encoding the UMI, a sequence complementary to the sequence encoding the TSO handle, a sequence complementary to the sequence encoding the sequence encoding a TSO hybridization site, and a sequence complementary to the target nucleic acid sequences.

76. The method of any one of claims 27-75, wherein sequencing comprises synthesizing a cDNA sequence comprising one of the complexed target nucleic acid sequences for each of the complexed target nucleic acid sequences.

77. The method of any one of claims 27-76, wherein the cDNA sequence comprises the target nucleic acid sequence, UMI, and individually unique chamber barcode sequence.

78. The method of any one of claims 27-77, wherein sequencing comprises removing the cDNA sequences from the chamber.

79. The method of any one of claims 27-78, wherein sequencing comprises amplifying the cDNA sequences by PCR.

80. The method of any one of claims 27-79, wherein the sequencing method is next generation sequencing (NGS).

81. The method of any one of claims 27-80, further comprising analyzing the cDNA sequences.

82. The method of any one of claims 27-81, further comprising analyzing the cDNA sequences.

83. The method of any one of claims 27-82, wherein the cDNA sequences are clustered by barcode sequence.

84. The method of any one of claims 27-83, wherein the cDNA sequences are quantified by bioanalyzer.

85. The method of any one of claims 27-84, wherein the target protein contacts the bead to form a CB-target protein complex.

86. The method of any one of claims 27-85, wherein the detecting the target protein comprises contacting the CB-target protein complex with a labeled secondary antibody and imaging the labeled secondary antibody.

87. The method of any one of the preceding claims, wherein the spatial resolution is 0.1 μm to 100 μm.

88. The method of any one of the preceding claims, wherein the spatial resolution is single cell.
